(19) United States
(12) Patent Application Publication
(10) Pub. No.: US 2022/0362177 A1
(43) Pub. Date: Nov. 17, 2022

(54) CERAMIDE RATIOS AS PREDICTIVE AND THERAPEUTIC BIOMARKERS FOR LEUKEMIAS

(71) Applicant: University of Virginia Patent Foundation, Charlottesville, VA (US)

(72) Inventors: Mark Kester, Afton, VA (US); Tye Deering, Charlottesville, VA (US); Thomas P. Loughran, JR., Charlottesville, VA (US); Todd E. Fox, Charlottesville, VA (US); Pedro Filipe Da Costa Pinheiro, Charlottesville, VA (US); David J. Feith, Palmyra, VA (US)

(73) Assignee: University of Virginia Patent Foundation, Charlottesville, VA (US)

(21) Appl. No.: 17/769,907

(22) PCT Filed: Oct. 16, 2020
(86) PCT No.: PCT/US2020/056110

§ 371 Date: Apr. 18, 2022

Related U.S. Application Data
(60) Provisional application No. 62/916,031, filed on Oct. 16, 2019.

(51) Int. Cl. A61K 31/164; G01N 33/92; A61K 31/704; A61K 31/635; A61K 31/706; A61K 31/444; A61K 31/53; A61K 31/553; A61K 31/497; A61K 38/16; A61P 35/02

(52) U.S. Cl. CPC A61K 31/164; G01N 33/92; A61K 31/704; A61K 31/635; A61K 31/706; A61K 31/444; A61K 31/53; A61K 31/553; A61K 31/497; A61K 38/164; A61P 35/02; G01N 2405/08; G01N 2800/56; G01N 2800/22

(57) ABSTRACT

Provided are methods for treating diseases, disorders, and conditions associated with undesirable cellular proliferation in subjects in need thereof. In some embodiments, the methods include administering to a subject in need thereof a therapeutically effective amount of a composition comprising, consisting essentially of, or consisting of a chemotherapeutic agent and a short chain ceramide. Also provided are methods for increasing total ceramide levels in cells, for increasing long chain ceramide to a very long chain ceramide ratios in cells, methods for enhancing apoptosis of cells, for prognosing subjects with diseases, disorders, and conditions associated with undesirable cellular proliferation with respect to treatments, for increasing sensitivities of drug-resistant tumor and/or cancer cells to chemotherapeutics, and compositions that have one or more short chain ceramides and one or more chemotherapeutically active agents.

### CROSS REFERENCE TO RELATED APPLICATION

The presently disclosed subject matter claims the benefit of U.S. Provisional Patent Application Ser. Nos. 62/916,031, filed Oct. 16, 2019, the disclosure of which is incorporated herein by reference in its entirety.

### GOVERNMENT INTEREST

This invention was made with government support under Grant No. 5-PO1-CA171983-06A1 awarded by National Cancer Institute. The government has certain rights in the invention.

### TECHNICAL FIELD

The presently disclosed subject matter relates in some embodiments to bioactive-lipid biomarkers that have predictive and therapeutic value. More particularly, the presently disclosed subject matter relates to methods for employing certain ceramide ratios as predictive and therapeutic biomarkers for leukemias.

### BACKGROUND

Acute Myeloid Leukemia (AML) is the most common acute leukemia in adults with a 5-year survival rate of only about 25% Despite a large number of clinical trials of both conventional and targeted therapies, there have been very few significant improvements in

AML outcome over the past 40 years. While outcomes for younger AML patients have modestly improved, the 5-year survival remain extremely poor for older patients at <5%. Though AML is a heterogeneous disease, standard of care (SOC) for fit and younger patients is initially intensive chemotherapy, including “7+3” cytarabine (AraC)/daunorubicin, followed by consolidation therapy. However, elderly or less fit patients are often unable to tolerate this regimen and thus may be treated with low intensity therapies with palliative intent. The FDA has recently approved several new AML therapeutics, including ivodesinib and enasidenib, which respectively target IDH1 and IDH2 mutations observed in 20% of AML, and VYXEOS™, a liposomal delivery system for daunorubicin and AraC. The multikinase inhibitor midostaurin was also recently approved for use in induction and consolidation along with standard chemotherapy for FLT3-mutated patients and several FLT3 inhibitors are in clinical trials, with gilteritinib recently receiving approval for FLT3-mutant relapsed or refractory AML (RR-AML).

Despite these new treatments, survival rates have changed little and virtually all patients develop RR-AML. Thus, there is a strong clinical need in AML to improve treatment of RR-AML patients. There is no consensus around a preferred intensive chemotherapy regimen for RR-AML, and the choice is based upon clinical experience and institutional approach. In order to determine the best treatment, the likelihood of disease progression need to be determined.

This presently disclosed subject matter thus relates in some embodiments to here-to-fore new bioactive-lipid biomarkers that have predictive and therapeutic value to clinicians to guide treatment options in diseases, disorders, and/or conditions associated with undesirable cellular proliferation, including but not limited to tumors and/or cancers such as leukemias.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, the presently disclosed subject matter relates to methods for treating diseases, disorders, and/or conditions associated with undesirable cellular proliferation. In some embodiments, the methods comprise, consist essentially of, or consist of administering to a subject in need thereof a therapeutically effective amount of a composition that comprises, consists essentially of, or consists of one or more chemotherapeutic agents and a short chain ceramide. In some embodiments, the disease, disorder, and/or condition associated with undesirable cellular proliferation is a tumor and/or a cancer. In some embodiments, the tumor and/or the cancer is a leukemia, optionally Acute Myeloid Leukemia (AML). In some embodiments, the chemotherapeutic agent is selected from the group consisting of daunorubicin, AraC, venetoclax, ivodesinib, enasidenib, midostaurin, gilteritinib, decitabine, azacitadine, a histone deacetylase (HDAC) inhibitor, an epigenetic regulator, a histone demethylase inhibitor, or any combination or subcombination thereof. In some embodiments, the composition comprises venetoclax in combination with one or more of daunorubicin, AraC, ivodesinib, enasidenib, midostaurin, gilteritinib, decitabine, azacitadine, a histone deacetylase (HDAC) inhibitor, an epigenetic regulator, and a histone demethylase inhibitor. In some embodiments, the composition comprises, consists essentially of, or consists of venetoclax and AraC, venetoclax and decitabine, or venatoclax and azacitadine. In some embodiments, composition comprises a ceramide nanoliposome (CNL) that is associated with decitibine, azacitadine, AraC, venetoclax, or any combination or subcombination thereof. In some embodiments, the composition comprises AraC, venetoclax, and one or more short chain ceramides.

In some embodiments, the presently disclosed subject matter also relates to methods for increasing total ceramide levels in cells. In some embodiments, the methods comprise, consist essentially of, or consist of contacting the cell with an effective amount of a composition comprising, consisting essentially of, or consisting of one or more short chain ceramides and one or more chemotherapeutic agents. In some embodiments, the chemotherapeutic agent is selected from the group consisting of daunorubicin, AraC, venetoclax, ivodesinib, enasidenib, midostaurin, gilteritinib, decitabine, azacitadine, a histone deacetylase (HDAC) inhibitor, an epigenetic regulator, a histone demethylase inhibitor, or any combination or subcombination thereof In some embodiments, the composition comprises venetoclax in combination with one or more of daunorubicin, AraC, ivodesinib, enasidenib, midostaurin, gilteritinib, decitabine, azacitadine, a histone deacetylase (HDAC) inhibitor, an epigenetic regulator, and a histone demethylase inhibitor.

In some embodiments, the composition comprises, consists essentially of, or consists of venetoclax and AraC. In some embodiments, composition comprises a ceramide nanoliposome (CNL) that is associated with decitibine, azacitadine, AraC, venetoclax, or any combination or subcombination thereof. In some embodiments, the composition comprises AraC, venetoclax, and one or more short chain ceramides.

In some embodiments, the presently disclosed subject matter also relates to methods for increasing a ratio of a long chain ceramide to a very long chain ceramide in a cell. In some embodiments, the methods comprise, consist essentially of, or consist of contacting the cell with an effective amount of a composition comprising, consisting essentially of, or consisting of one or more short chain ceramides and a chemotherapeutic agent. In some embodiments, the long chain ceramide is a C16 and/or a C18 ceramide. In some embodiments, the very long chain ceramide is a C24 ceramide. In some embodiments, the chemotherapeutic agent is selected from the group consisting of daunorubicin, AraC, venetoclax, ivodesinib, enasidenib, midostaurin, gilteritinib, decitabine, azacitadine, a histone deacetylase (HDAC) inhibitor, an epigenetic regulator, and a histone demethylase inhibitor, or any combination or subcombination thereof In some embodiments, the composition comprises venetoclax in combination with one or more of daunorubicin, AraC, ivodesinib, enasidenib, midostaurin, gilteritinib, decitabine, azacitadine, a histone deacetylase (HDAC) inhibitor, an epigenetic regulator, and a histone demethylase inhibitor. In some embodiments, the composition comprises, consists essentially of, or consists of venetoclax and AraC, venetoclax and decitabine, or venatoclax and azacitadine. In some embodiments, the composition comprises a ceramide nanoliposome (CNL) that is associated with decitibine, azacitadine, AraC, venetoclax, or any combination or subcombination thereof. In some embodiments, the composition comprises one or more short chain ceramides and AraC or venetoclax or both AraC and venetoclax. In some embodiments, the cell is a tumor and/or a cancer cell, optionally a leukemia cell, further optionally an AML cell. In some embodiments, the presently disclosed methods further comprises contacting the cell with a further anti-leukemia therapeutic agent.

The presently disclosed subject matter also relates in some embodiments to methods for enhancing apoptosis of cells in which apoptosis is desirable. In some embodiments, the methods comprise, consist essentially of, or consist of contacting the cell with a composition comprising, consisting essentially of, or consisting of one or more chemotherapeutic agents in combination with one or more small chain ceramides. In some embodiments, the one or more chemotherapeutic agents are selected from the group consisting of daunorubicin, AraC, venetoclax, ivodesinib, enasidenib, midostaurin, gilteritinib, decitabine, azacitadine, a histone deacetylase (HDAC) inhibitor, an epigenetic regulator, and a histone demethylase inhibitor, or any combination or subcombination thereof In some embodiments, the composition comprises venetoclax in combination with one or more of daunorubicin, AraC, ivodesinib, enasidenib, midostaurin, gilteritinib, decitabine, azacitadine, a histone deacetylase (HDAC) inhibitor, an epigenetic regulator, and a histone demethylase inhibitor. In some embodiments, the composition comprises, consists essentially of, or consists of venetoclax and AraC, venetoclax and decitabine, or venatoclax and azacitadine. In some embodiments, the composition comprises a ceramide nanoliposome (CNL) that is associated with decitibine, azacitadine, AraC, venetoclax, or any combination or subcombination thereof. In some embodiments, the composition comprises one or more short chain ceramides and AraC or venetoclax or both AraC and venetoclax. In some embodiments, the cell is a tumor and/or a cancer cell, optionally a leukemia cell, further optionally an AML, cell. In some embodiments. the contacting increases a C16 and/or C18 ceramide to C24 ceramide ratio in the cell. In some embodiments, the contacting increases a C16 ceramide to C24 ceramide ratio, a C18 ceramide to C24 ceramide ratio, or both in the cell. In some embodiments, the contacting decreases a C16 ceramide to C24 ceramide ratio, a C18 ceramide to C24 ceramide ratio, or both in the cell. In some embodiments, one or ore of the C16, C18, and/or C24 ceramides is a saturated ceramide. In some embodiments, one or ore of the C16, C18, and/or C24 ceramides is a monounsaturated ceramide.

In some embodiments, the presently disclosed subject matter also relates to methods for prognosing subject with a disease, disorder, and/or condition associated with undesirable cellular proliferation with respect to a treatment. In some embodiments, the methods comprise, consist essentially of, or consist of determining a ratio of long chain ceramide to very long chain ceramide in the subject, wherein the ratio is indicative of progression-fee survival (PFS), improved overall survival, complete remission, or any combination thereof in the subject. In some embodiments, the disease, disorder, and/or condition associated with undesirable cellular proliferation is a tumor and/or a cancer. In some embodiments, the tumor and/or the cancer is a leukemia, optionally Acute Myeloid Leukemia (AML). In some embodiments, the long chain ceramide is selected from the group consisting of a C16 ceramide, a C18 ceramide, or any combination thereof. In some embodiments, the very long chain ceramide is a C24 ceramide, optionally a C24:1 ceramide. In some embodiments, a high C16 and/or C18 to C24 ceramide ratio in the subject is indicative of improved survival. In some embodiments, a low C16 and/or C18 to C24 lactosylceramide ratio in the subject is indicative of improved survival. In some embodiments, the composition comprises a ceramide nanoliposome (CNL). In some embodiments, the result of the determining step is employed to initiate, continue, modify, or terminate the treatment.

In some embodiments, the presently disclosed subject matter also relates to methods for increasing sensitivity of a drug-resistant tumor and/or cancer cell to a chemotherapeutic. In some embodiments, the method comprises, consists essentially of, or consists of contacting the drug-resistant tumor and/or cancer cell with a therapeutically effective amount of a composition comprising, consisting essentially of, or consisting of one or more small chain ceramides in combination with the chemotherapeutic, wherein the sensitivity of the drug-resistant tumor and/or cancer cell to the chemotherapeutic is increased relative to the sensitivity of the drug-resistant tumor and/or cancer cell prior to the contacting. In some embodiments, the chemotherapeutic is venetoclax, AraC, decitabine, azacitadine, an HDAC inhibitor, an epigenetic regulator, a histone demethylase inhibitor, or any combination of subcombination thereof. In some embodiments, the contacting increases a ratio of C16 and/or C18 ceramide to C24 ceramide in the drug-resistant tumor and/or cancer cell. In some embodiments, the tumor and/or cancer cell is present in a subject and the composition is administered to the subject via a route and in an amount sufficient to increase the sensitivity of the drug-resistant tumor and/or cancer cell to the chemotherapeutic.

In some embodiments of the presently disclosed methods, the subject is a mammal, optionally a human.

In some embodiments, the presently disclosed subject matter also relates to compositions, optionally pharmaceutical compositions, that comprise, consisting essentially of, or consist of one or more short chain ceramides and one or more chemotherapeutically active agents. In some embodiments, the composition further comprises one or more pharmaceutically acceptable carriers, diluents, and/or excipients, optionally wherein the composition is pharmaceutically acceptable for use in a human. In some embodiments, the one or more short chain ceramides are saturated C6 ceramides, monosaturated C6 ceramides, or any combination thereof. In some embodiments, the one or more chemotherapeutically active agents are selected from the group consisting of venetoclax, AraC, decitabine, azacitadine, an HDAC inhibitor, an epigenetic regulator, a histone demethylase inhibitor, and combinations thereof.

In some embodiments, the presently disclosed subject matter also relates to composition for use in the methods of the presently disclosed subject matter. Thus, in some embodiments the presently disclosed subject matter relates to compositions for us in a method for treating a disease, disorder, and/or condition associated with undesirable cellular proliferation, for use in increasing total ceramide levels in a cell, for use in increasing a ratio of a long chain ceramide to a very long chain ceramide in a cell, for use in enhancing apoptosis of a cell in which apoptosis is desirable, for prognosing a subject with a disease, disorder, and/or condition associated with undesirable cellular proliferation with respect to a treatment, and/or for use in increasing sensitivity of a drug-resistant tumor and/or cancer cell to a chemotherapeutic, the composition comprising, consisting essentially of, or consisting of a chemotherapeutic agent in combination with one or more small chain ceramides. In some embodiments, the disease, disorder, and/or condition associated with undesirable cellular proliferation is a tumor and/or a cancer, optionally a leukemia, further optionally Acute Myeloid Leukemia (AML). In some embodiments, the chemotherapeutic agent is selected from the group consisting of daunorubicin, AraC, venetoclax, ivodesinib, enasidenib, midostaurin, gilteritinib, decitabine, azacitadine, a histone deacetylase (HDAC) inhibitor, an epigenetic regulator, a histone demethylase inhibitor, or any combination or subcombination thereof. In some embodiments, the composition comprises venetoclax in combination with one or more of daunorubicin, AraC, ivodesinib, enasidenib, midostaurin, gilteritinib, decitabine, azacitadine, a histone deacetylase (HDAC) inhibitor, an epigenetic regulator, and a histone demethylase inhibitor. In some embodiments, the composition comprises, consists essentially of, or consists of venetoclax and AraC, venetoclax and decitabine, or venatoclax and azacitadine. In some embodiments, the composition comprises a ceramide nanoliposome (CNL) that is associated with decitibine, azacitadine, AraC, venetoclax, or any combination or subcombination thereof. In some embodiments, the composition comprises AraC, venetoclax, and one or more short chain ceramides. In some embodiments, the long chain ceramide is a C16 and/or a C18 ceramide. In some embodiments, the very long chain ceramide is a C24 ceramide, optionally a C24:1 ceramide. In some embodiments, the composition increases a C16 and/or C18 ceramide to C24 ceramide ratio in the cell. In some embodiments, the composition increases a C16 ceramide to C24 ceramide ratio, a C18 ceramide to C24 ceramide ratio, or both in the cell. In some embodiments, the composition decreases a C16 ceramide to C24 ceramide ratio, a C18 ceramide to C24 ceramide ratio, or both in the cell. In some embodiments, the C24 ceramide is a C24:1 ceramide.

Accordingly, it is an object of the presently disclosed subject matter to provide compositions and methods for treating diseases, disorders, and/or conditions associated with undesirable cellular proliferation.

This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description and Figures.

### BRIEF DESCRIPTIONS OF THE FIGURES

FIG. 1. Ceramide is generated from three main pathways (sphingomyelin hydrolysis, de novo synthesis, or salvage pathway), whereas the degradation of ceramide is facilitated by four main enzyme families (SMS, GCS, CDase, and CerK). In normal cells, these pathways are stable. However, in diseases, disorders, and/or conditions associated with undesirable cellular proliferation such as but not limited to leukemia, as exemplified by AML, similarly to increasing flow through the faucet or clogging the drain, these pathways are altered leading to aberrant phenotype. The presently disclosed subject matter relates in some embodiments to the demonstration that elevating pro-apoptotic ceramide species is therapeutically efficacious for AML.

FIG. 2. TCGA AML RNA-seq data indicated widespread disruption of sphingolipid metabolism gene expression (rows) across AML molecular subtypes (columns, arranged into mutation functional groups). There is a profound upregulation (red in a color version of the Figure; dark gray in the grayscale version of the Figure) of pro-survival sphingolipid metabolism genes (yellow in a color version of the Figure; medium gray in the grayscale version of the Figure) and similarly downregulation (green in a color version of the Figure; light gray in the grayscale version of the Figure) of pro-death sphingolipid metabolism.

FIGS. 3A-3F. Sphingolipid Perturbations Correspond to AML Patient Survival. Project correlative studies demonstrated that (FIG. 3A) high acid ceramidase (AC) activity (n=63, p=0.03), (FIG. 3B) high SPHK1 expression (n=41), and (FIG. 3C) high GCS expression (n=41) were all associated with reduced overall or disease-free survival. Progression-free survival (n=39) was improved with (FIG. 3D) high C18/C24:1 ceramide ratios, (FIG. 3E) high C16/C24:1 ceramide ratios, and (FIG. 3F) low C18/C24:1-lactosylceramide ratios.

FIG. 4. Ceramide chain length altered biological and biophysical responses. MOLM-14 cells were treated with the indicated ceramides for 24 hours and viability was assessed by MTS.

FIGS. 5A and 5B. FIG. 5A: Ceramide levels from MOLM-14 cells transduced with a control vector or CerS6 for overexpression and treated with Ghost or CNL. (n=5; *or #=significant to Cnt, ¥=significant to CerS6, ‡=significant to Cnt+CNL). FIG. 5B: Apoptosis was assessed in control and CerS6 overexpressing MOLM-14 after 72 hours with the indicated treatments (n=3; *=significant difference observed between vector control and CerS6 overexpression).

FIGS. 6A-6H. CAV treatment is efficacious in both AML cell lines and primary AML culture. FIGS. 6A-6C: Cell lines were treated with the listed treatment for 24 hours and subjected to the MTS viability assay (n=5; *=significant to Vehicle). FIG. 6D: Cryopreserved human AML patient cells (n=6) cultured and treated for 24 hours with ceramide nanoliposome (CNL), cytarabine (AraC), venetoclax (VEN) or the combinations. Post-treatment, the cells were propagated for 10-14 days, and blast colonies counted (Data are the mean±SD. *P<0.05, **P<0.01). Augmented long-chain ceramide formation with CAV Trial regimen may mediate AML cell death. Cells were treated as indicated for 24 hours prior to lipid analyses. Shown are total ceramide levels (FIG. 6E) and individual ceramide species (FIGS. 6F and 6G). The ratios of the change in long chain C16 to very long chain C24:1 ceramide is shown in FIG. 7H (n=5; *=significant to Cnt).

FIGS. 7A-7I. CAV treatment alters expression of genes involved in cell survival relevant to AraC and Venetoclax resistance mechanism. MOLM-14 cells were treated with the listed conditions for 24 hours and protein harvested. Western blots were performed and quantitated via densitometry. Representative blots are shown in FIG. 7A and quantification in FIGS. 7B-7I. *: significant to Vehicle.

FIGS. 8A and 8B: CAV treatment increases survival in MOLM-13 AML animal model. FIG. 8A: Bioluminescence images of MOLM-13 Luc-YFP bearing NRG mice treated with CNL (29.1 mg/kg; IV; every other day), AraC (25 mg/kg; IP; daily), and venetoclax (100 mg/kg; PO; daily).

FIG. 8C: Survival curves of the indicated treatment groups. n=4-8/group. *=statistical difference between VEN+AraC and CNL+VEN+AraC. FIG. 8C: Survival of C1498-bearing C57BL/6J mice were treated with either Control (Lip-Ghost), CNL (31.2 mg/kg), Cytarabine (75 mg/kg) or CNL+Cytarabine. The CNL+Cytarabine group had median survival time of 55 days compared with 41 days for the Cytarabine mono-therapy group (p=0.045), and 31 days for Control group (p=0.0005).

### DETAILED DESCRIPTION

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition, and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell is a cell being examined.

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, the terms “condition”, “disease condition”, “disease”, “disease state”, and “disorder” refer to physiological states in which diseased cells or cells of interest can be targeted with the compositions of the presently disclosed subject matter. In some embodiments, a disease is leukemia, which in some embodiments is Acute Myeloid Leukemia (AML).

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

A “fragment”, “segment”, or “subsequence” is a portion of an amino acid sequence, comprising at least one amino acid, or a portion of a nucleic acid sequence comprising at least one nucleotide. The terms “fragment”, “segment”, and “subsequence” are used interchangeably herein.

As used herein, a “functional” biological molecule is a biological molecule in a form in which it exhibits a property by which it can be characterized. A functional enzyme, for example, is one that exhibits the characteristic catalytic activity by which the enzyme can be characterized.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichythyes (bony fish), Amphibia (amphibians), Reptilia (reptiles), Ayes (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments, humans.

As used herein, a “subject in need thereof” is a patient, animal, mammal, or human, who will benefit from the method of this presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

### II. Exemplary Methods

In some embodiments, the presently disclosed subject matter provides methods for treating a disease, disorder, and/or condition associated with undesirable cellular proliferation comprising, consisting essentially of, or consisting of administering to a subject in need thereof a therapeutically effective amount of a composition that comprises, consists essentially of, or consists of a chemotherapeutic agent and a short chain ceramide.

As used herein, the phrase “disease, disorder, and/or condition associated with undesirable cellular proliferation” relates to any disease, disorder, and/or condition at least one symptom or consequence of which relates to or results from cellular proliferation that is in excess of which would normally occur in the absence of the disease, disorder, and/or condition. Exemplary diseases, disorders, and/or conditions associated with undesirable cellular proliferation include, but are not limited to tumors and/or cancers, which can include leukemia, optionally Acute Myeloid Leukemia (AML) and/or Relapsed/Refractory acute myeloid leukemia (RR-AML).

RR-AML is a particularly difficult therapeutic challenge. Different chemotherapy regimens can be employed in an attempt to achieve disease remission prior to immune cell and/or stem cell transplantation. There is currently no universally accepted treatment regimen, although various approaches such as CLAG-M (cladribine, cytarabine, mitoxantrone, and filgrastim), FLAG (fiudarabine, cytarabine, idarubicin, and filgrastim), or MEC (mitoxantrone, etoposide, and cytarabine) have been used.

A retrospective study compared two commonly used regimens in RR-AML: CLAG (cladribine, cytarabine, and filgrastim) and MEC (mitoxantrone, etoposide & cytarabine). See Price et al. (2011) Salvage chemotherapy regimens for acute myeloid leukemia: Is one better? Efficacy comparison between CLAG and MEC regimens. Leukemia Research 35(3):301-304. Complete response rates of 37.9% for CLAG (n=97) and 23.8% for MEC (n=65) (p=0.048) were reported, with a median overall survival of 7.3 and 4.5 months, respectively (p=0.05). In primary refractory disease, the complete response rate was 45.5% for CLAG and 22.2% for MEC (p=0.09), with a median overall survival of 11 and 4.5 months, respectively (p=0.07). In patients with relapsed AML, the complete response rate was 36.8% with CLAG and 25.9% with MEC (p=0.35) and the median overall survival was 6.7 and 6.7 months, respectively (p=0.87). The combination of the purine nucleoside analogue (cladribine) with cytarabine increases the intracellular accumulation of Ara-C-5′ triphosphate (ara-C TP) that causes cytotoxicity in leukemic blasts. Addition of granulocyte-colony stimulating factor (G-CSF) further improves the effects of a purine nucleoside analogue in combination with Ara-C by activating the leukemic cells and making them susceptible to chemotherapy's effect. It is clear, however, that these approaches are not efficacious for many AML and RR-AML patients.

As disclosed herein, short chain ceramides can enhance the activities of various chemotherapeutic agents by modulating ceramide biosynthesis and/or degradation in cells, tissues, and organs. As used herein, the phrase “short chain ceramide” refers to a ceramide that in some embodiments has a backbone of from 2-10 carbons (i.e., C2-C10 ceramides). This is in contrast to the long chain ceramides and very long chain ceramides, which have backbones of from 12-20 carbons (i.e., C12-C20 ceramides) and of at least 22 carbons (e.g., C22, C24, C26, C28, and C30 ceramides), respectively. As would understood by one of ordinary skill in the art, ceramides can be saturated or monounsaturated, and the term “ceramide” as used herein encompasses both saturated and monounsaturated forms of each individual ceramide species.

The effects of short chain ceramides with respect to enhancing the activities of chemotherapeutic agents are not limited to particular chemotherapeutics, and as such, any chemotherapeutic agent can be employed with the short chain ceramides of the presently disclosed subject matter. Thus, in some embodiments a chemotherapeutic agent can be an mTOR inhibitors (including, but are not limited to, CCI-779, rapamycin, temsirolimus, everolimus, RAD001 and AP-23573), an HSP-90 inhibitor (including, but are not limited to, geldanamycin, radicicol, 17-AAG, KOS-953, 17-DMAG, CNF-101, CNF-1010, 17-AAG-nab, NCS-683664, efungumab, CNF-2024, PU3, PU24FC1, VER-49009, IPI-504, SNX-2112 and STA-9090), an HDAC inhibitor (including, but are not limited to, suberoylanilide hydroxamic acid (SAHA), MS-275, valproic acid, TSA, LAQ-824, trapoxin and depsipeptide), a MEK inhibitor (including, but are not limited to, PD-325901, ARRY-142886, ARRY-438162 and PD-98059), a CDK inhibitor (including, but are not limited to, flavopyridol, MCS-5A, CVT-2584, seliciclib ZK-304709, PHA-690509, BMI-1040, GPC-286199, BMS-387032, PD-332991 and AZD-5438), an alkylating agent (including, but are not limited to, nitrogen mustard N-oxide, cyclophosphamide, ifosfamide, trofosfamide, chlorambucil, melphalan, busulfan, mitobronitol, carboquone, thiotepa, ranimustine, nimustine, Cloretazine™ (laromustine), AMD-473, altretamine, AP-5280, apaziquone, brostallicin, bendamustine, carmustine, estramustine, fotemustine, glufosfamide, KW-2170, mafosfamide, mitolactol, lomustine, treosulfan, dacarbazine and temozolomide), an antimetabolite (including, but are not limited to, methotrexate, 6-mercaptopurine riboside, mercaptopurine, 5-fluorouracil (5-FU) alone or in combination with leucovorin, tegafur, UFT, doxifluridine, carmofur, cytarabine, cytarabine ocfosfate, enocitabine, S-1, pemetrexed, gemcitabine, fludarabine, 5-azacitidine, capecitabine, cladribine, clofarabine, decitabine, eflornithine, ethenylcytidine, cytosine arabinoside, hydroxyurea, TS-1, melphalan, nelarabine, nolatrexed, disodium pemetrexed, pentostatin, pelitrexol, raltitrexed, triapine, trimetrexate, vidarabine, mycophenolic acid, ocfosfate, pentostatin, tiazofurin, ribavirin, EICAR, hydroxyurea and deferoxamine), a topoisomerase inhibiting agent (including, but are not limited to, aclarubicin, amonafide, belotecan, camptothecin, 10-hydroxycamptothecin, 9-amino-camptothecin, amsacrine, dexrazoxane, diflomotecan, irinotecan HC1, edotecarin, epirubicin, etoposide, exatecan, becatecarin, gimatecan, lurtotecan, orathecin, BN-80915, mitoxantrone, pirarbicin, pixantrone, rubitecan, sobuzoxane, SN-38, tafluposide and topotecan), and antibody (including, but are not limited to, rituximab, cetuximab, bevacizumab, trastuzumab, CD40-specific antibodies and IGF1R-specific antibodies, chTNT-1/B, denosumab, edrecolomab, WX G250, zanolimumab, lintuzumab and ticilimumab), a plant alkaloid (including, but are not limited to, vincristine, vinblastine, vindesine and vinorelbine), a proteasome inhibitor (including, but are not limited to, bortezomib, MG-132, NPI-0052 and PR-171), an immunological (including, but are not limited to, interferons and numerous other immune-enhancing agents. Interferons include interferon alpha, interferon alpha-2a, interferon alpha-2b, interferon beta, interferon gamma-1a, interferon gamma-lb, interferon gamma-nl and combinations thereof. Other agents include filgrastim, lentinan, sizofilan, BCG live, ubenimex, WF-10 (tetrachlorodecaoxide or TCDO), aldesleukin, alemtuzumab, BAM-002, dacarbazine, daclizumab, denileukin, gemtuzumab ozogamicin, ibritumomab, imiquimod, lenograstim, melanoma vaccine, molgramostim, sargramostim, tasonermin, teceleukin, thymalfasin, tositumomab, VIRULIZIN™ immunotherapeutic of Lorus Pharmaceuticals, Z-100 (specific substance of Maruyama or SSM), ZEVALIN™ (90Y-ibritumomab tiuxetan), epratuzumab, mitumomab, oregovomab, pemtumomab, PROVENGE™ (sipuleucel-T), teceleukin, THEROCYS™ (Bacillus Calmette-Guerin), cytotoxic lymphocyte antigen 4 (CTLA4) antibodies and agents capable of blocking CTLA4 such as MDX-010), a pyrimidine analog (including, but are not limited to, 5-fluorouracil, floxuridine, doxifluridine, raltitrexed, cytarabine, cytosine arabinoside, fludarabine, triacetyluridine, troxacitabine and gemcitabine), a purine analog (including, but are not limited to, mercaptopurine and thioguanine), a antimitotic agent (including, but are not limited to, N-(2-((4-hydroxyphenyl)amino)pyridin-3 -yl)-4-methoxybenzenesulfonamide, paclitaxel, docetaxel, larotaxel, epothilone D, PNU-100940, batabulin, ixabepilone, patupilone, XRP-9881, vinflunine and ZK-EPO (synthetic epothilone), or any combination thereof. In some embodiments the chemotherapeutic agent is selected from the group consisting of daunorubicin, AraC, venetoclax, ivodesinib, enasidenib, midostaurin, gilteritinib, decitabine, azacitadine, a histone deacetylase (HDAC) inhibitor, an epigenetic regulator, a histone demethylase inhibitor, or any combination or subcombination thereof. In some embodiments, the composition comprises venetoclax in combination with one or more of daunorubicin, AraC, ivodesinib, enasidenib, midostaurin, gilteritinib, decitabine, azacitadine, a histone deacetylase (HDAC) inhibitor, an epigenetic regulator, and a histone demethylase inhibitor. In some embodiments, the composition comprises, consists essentially of, or consists of venetoclax and AraC, venetoclax and decitabine, or venatoclax and azacitadine. In some embodiments, the composition comprises AraC, venetoclax, and one or more short chain ceramides. In some embodiments, the short chain ceramide is a C6 ceramide.

In some embodiments, the composition comprises a ceramide nanoliposome (CNL) that is associated with decitibine, azacitadine, AraC, venetoclax, or any combination or subcombination thereof. The preparation and use of CNLs is described, for example, in U.S. Pat. No. 8,747,891 and U.S. Patent Application Publication Nos. 2020/0170970, 2020/0268665, each of which is incorporated herein by reference in its entirety.

In some embodiments, the presently disclosed subject matter also provides methods for increasing total ceramide levels in cells. In some embodiments, the methods comprise, consist essentially of, or consist of contacting the cell with an effective amount of a composition comprising, consisting essentially of, or consisting of one or more short chain ceramides and one or more chemotherapeutic agents, optionally wherein the one or more chemotherspeutic agents are selected from the group consisting of daunorubicin, AraC, venetoclax, ivodesinib, enasidenib, midostaurin, gilteritinib, decitabine, azacitadine, a histone deacetylase (HDAC) inhibitor, an epigenetic regulator, a histone demethylase inhibitor, and combinations thereof.

In some embodiments, the presently disclosed subject matter also provides methods for increasing a ratio of a long chain ceramide to a very long chain ceramide in a cell, the method comprising contacting the cell with an effective amount of a composition comprising, consisting essentially of, or consisting of one or more short chain ceramides and a chemotherapeutic agent. In some embodiments, the long chain ceramide is a C16 ceramide, a C18 ceramide, or a combination thereof. In some embodiments, the very long chain ceramide is a C24 ceramide, optionally a C24:1 ceramide. In some embodiments, chemotherapeutic agent is selected from the group consisting of daunorubicin, AraC, venetoclax, ivodesinib, enasidenib, midostaurin, gilteritinib, decitabine, azacitadine, a histone deacetylase (HDAC) inhibitor, an epigenetic regulator, a histone demethylase inhibitor, or any combination or subcombination thereof In some embodiments, the composition comprises venetoclax in combination with one or more of daunorubicin, AraC, ivodesinib, enasidenib, midostaurin, gilteritinib, decitabine, azacitadine, a histone deacetylase (HDAC) inhibitor, an epigenetic regulator, and a histone demethylase inhibitor. In some embodiments, the composition comprises, consists essentially of, or consists of venetoclax and AraC, venetoclax and decitabine, or venatoclax and azacitadine. In some embodiments, the composition comprises a ceramide nanoliposome (CNL) that is associated with decitibine, azacitadine, AraC, venetoclax, or any combination or subcombination thereof. In some embodiments, the composition comprises one or more short chain ceramides and AraC or venetoclax or both AraC and venetoclax.

In some embodiments of the presently disclosed methods, the cell is a tumor and/or a cancer cell, optionally a leukemia cell, further optionally an AML cell. In some embodiments, the presently disclosed methods further comprise, consist essentially of, or consist of contacting the cell with a further anti-leukemia therapeutic agent.

The presently disclosed subject matter also provides methods for enhancing apoptosis of cells in which apoptosis is desirable. In some embodiments, the methods comprise, consist essentially of, or consist of contacting the cell with a composition comprising, consisting essentially of, or consisting of a chemotherapeutic agent in combination with one or more small chain ceramides. In some embodiments, the chemotherapeutic agent is selected from the group consisting of daunorubicin, AraC, venetoclax, ivodesinib, enasidenib, midostaurin, gilteritinib, decitabine, azacitadine, a histone deacetylase (HDAC) inhibitor, an epigenetic regulator, a histone demethylase inhibitor, or any combination or subcombination thereof. In some embodiments, composition comprises venetoclax in combination with one or more of daunorubicin, AraC, ivodesinib, enasidenib, midostaurin, gilteritinib, decitabine, azacitadine, a histone deacetylase (HDAC) inhibitor, an epigenetic regulator, and a histone demethylase inhibitor. In some embodiments, the composition comprises, consists essentially of, or consists of venetoclax and AraC, venetoclax and decitabine, or venatoclax and azacitadine. In some embodiments, the composition comprises a ceramide nanoliposome (CNL) that is associated with decitibine, azacitadine, AraC, venetoclax, or any combination or subcombination thereof. In some embodiments, the composition comprises one or more short chain ceramides and AraC or venetoclax or both AraC and venetoclax. In some embodiments, the cell is a tumor and/or a cancer cell, optionally a leukemia cell, further optionally an AML cell.

In some embodiments of the presently disclosed methods, the contacting increases a C16 and/or C18 ceramide to C24 ceramide ratio in the cell. In some embodiments, the contacting increases a C16 ceramide to C24 ceramide ratio, a C18 ceramide to C24 ceramide ratio, or both in the cell. In some embodiments, the increase is measured relative to the cell prior to and/or in the absence of the contacting step.

Alternatively or in addition, in some embodiments the contacting decreases a C16 ceramide to C24 ceramide ratio, a C18 ceramide to C24 ceramide ratio, or both in the cell. In some embodiments, the decrease is measured relative to the cell prior to and/or in the absence of the contacting step.

In some embodiments of the presently disclosed methods, the C16 ceramide, the C18 ceramide, or both are saturated ceramides (e.g., C16:0 and/or C18:0 ceramides). In some embodiments, the C24 ceramide is a C24:1 ceramide.

In some embodiments, the presently disclosed subject matter also provides methods for prognosing subjects with a disease, disorder, and/or condition associated with undesirable cellular proliferation with respect to treatments. In some embodiments, the methods comprise, consist essentially of, or consist of determining a ratio of long chain ceramide to very long chain ceramide in the subject, wherein the ratio is indicative of progression-fee survival (PFS), improved overall survival, complete remission, or any combination thereof in the subject. In some embodiments, the disease, disorder, and/or condition associated with undesirable cellular proliferation is a tumor and/or a cancer. In some embodiments, the tumor and/or the cancer is a leukemia, optionally Acute Myeloid Leukemia (AML). In some embodiments, the long chain ceramide is selected from the group consisting of a C16 ceramide, a C18 ceramide, or any combination thereof. In some embodiments, the very long chain ceramide is a C24 ceramide, optionally a C24:1 ceramide.

In some embodiments of the presently disclosed methods, a high C16 and/or C18 to C24 ceramide ratio in the subject is indicative of improved survival. In some embodiments, a low C16 and/or C18 to C24 lactosylceramide ratio in the subject is indicative of improved survival. As used herein in the context of ceramide and/or lactosylceramide ratios, the terms “high” and “low ” refer to ratios of long chain ceramides to very long chain ceramides. In normal subjects, the ratio of C16 to C24 (in some embodiments, C24:1) ceramides ranges from about 0.5:1 to about 1.5:1. Thus, in the context of a C16 to C24 ratio, a low ratio is in some embodiments less than about 1.0:1, in some embodiments less than about 0.9:1, in some embodiments less than about 0.8:1, in some embodiments less than about 0.7:1, in some embodiments less than about 0.6:1, and in some embodiments less than about 0.5:1. Similarly, in the context of a C16 to C24 ratio, a high ratio is in some embodiments greater than about 1.0:1, in some embodiments greater than about 1.1:1, in some embodiments greater than about 1.2:1, in some embodiments greater than about 1.3:1, in some embodiments greater than about 1.4:1, and in some embodiments greater than about 1.5:1.

In normal subjects, the ratio of C18 to C24 (in some embodiments, C24:1) ceramides or the ratio of C18 to C24 (in some embodiments, C24:1) lactosylceramides ranges from about 0.1:1 to about 0.7:1. Thus, in the context of a C18 to C24 (in some embodiments, C24:1) ceramide or lactosylceramide ratio, a low ratio is in some embodiments less than about 0.35:1, in some embodiments less than about 0.3:1, in some embodiments less than about 0.25, in some embodiments less than about 0.2:1, in some embodiments less than about 0.15:1, and in some embodiments less than about 0.1:1. Similarly, in the context of a C18 to C24 (in some embodiments, C24:1) ceramide or lactosylceramide ratio, a high ratio is in some embodiments greater than about 0.35:1, in some embodiments greater than about 0.4:1, in some embodiments greater than about 0.45:1, in some embodiments greater than about 0.5:1, in some embodiments greater than about 0.55:1, in some embodiments greater than about 0.6:1, in some embodiments greater than about 0.65:1, and in some embodiments greater than about 0.7:1.

In some embodiments of the presently disclosed methods, the composition comprises a ceramide nanoliposome (CNL).

The presently disclosed subject matter also provides in some embodiments methods for increasing sensitivity of drug-resistant tumors and/or cancer cells to chemotherapeutics. In some embodiments, the methods comprise, consist essentially of, or consist of contacting a drug-resistant tumor and/or cancer cell with a therapeutically effective amount of a composition comprising, consisting essentially of, or consisting of one or more small chain ceramides in combination with the chemotherapeutic, wherein the sensitivity of the drug-resistant tumor and/or cancer cell to the chemotherapeutic is increased relative to the sensitivity of the drug-resistant tumor and/or cancer cell prior to the contacting. In some embodiments, the chemotherapeutic is venetoclax, AraC, decitabine, azacitadine, an HDAC inhibitor, an epigenetic regulator, a histone demethylase inhibitor, or any combination of subcombination thereof. In some embodiments, the contacting increases a ratio of C16 and/or C18 ceramide to C24 ceramide in the drug-resistant tumor and/or cancer cell. In some embodiments, the tumor and/or cancer cell is present in a subject and the composition is administered to the subject via a route and in an amount sufficient to increase the sensitivity of the drug-resistant tumor and/or cancer cell to the chemotherapeutic.

In some embodiments, the methods of the presently disclosed subject matter are appropriate for use in subjects. As used herein, a “subject” of analysis, diagnosis, and/or treatment is an animal. Such animals include mammals. In some embodiments, a subject is a human. As used herein, a “subject in need thereof” is a patient, animal, mammal, or human, who will benefit from the methods and compositions of the presently disclosed subject matter. Thus, in some embodiments the subject is a mammal, optionally a human.

### III. Exemplary Compositions

### III.A. Generally

The presently disclosed subject matter also provides in some embodiments compositions, optionally pharmaceutical compositions, that comprise, consist essentially of, or consist of one or more short chain ceramides (including but not limited to C6 ceramides) and one or more chemotherapeutically active agents. In some embodiments, the one or more short chain ceramides are saturated C6 ceramides, monosaturated C6 ceramides, or any combination thereof. In some embodiments, the one or more chemotherapeutically active agents are selected from the group consisting of venetoclax, AraC, decitabine, azacitadine, an HDAC inhibitor, an epigenetic regulator, a histone demethylase inhibitor, and combinations thereof.

In some embodiments, the composition is administered as part of a nanoscale or microscale delivery vehicle, wherein the delivery vehicle is optionally selected from the group consisting of a liposome, a lipo/polymer, a microparticle, and a nanoparticle, or any combination thereof. In some embodiments, the delivery vehicle comprises a nanoliposome, wherein the nanoliposome encompasses the one or more short chain ceramides (including but not limited to C6 ceramides) and one or more chemotherapeutically active agents and/or comprises a lipid bilayer that comprises the one or more short chain ceramides (including but not limited to C6 ceramides) and one or more chemotherapeutically active agents.

In some embodiments, the delivery vehicle is designed to degrade in the subject in order to release the one or more short chain ceramides (including but not limited to C6 ceramides) and one or more chemotherapeutically active agents to the subject over a period of time. In some embodiments, the delivery vehicle releases the one or more short chain ceramides (including but not limited to C6 ceramides) and one or more chemotherapeutically active agents to the subject's circulation and/or a cell, tissue, and/or organ of subject over the period of time. In some embodiments, the delivery vehicle is designed to degrade subsequent to contact with the subject's digestive system or circulatory system. In some embodiments, the delivery vehicle is designed to degrade in the subject to release at least about 50% of the one or more short chain ceramides (including but not limited to C6 ceramides) and one or more chemotherapeutically active agents over a period of time of at least 30 minutes, at least 1 hour, at least 6 hours, at least 12 hours, at least 24 hours, or longer than 24 hours.

In some embodiments, the presently disclosed subject matter provides for the use of compositions comprising liposomes. Liposomes can be prepared by any of a variety of techniques that are known in the art. See e.g., Betageri et al. (1993) Liposome Drug Delivery Systems, Technomic Publishing, Lancaster, Pa., United States of America; Gregoriadis, ed. (1993) Liposome Technology, CRC Press, Boca Raton, Fla., United States of America; Janoff, ed. (1999) Liposomes: Rational Design, M. Dekker, New York, N.Y., United States of America; Lasic & Martin (1995) Stealth Liposomes, CRC Press, Boca Raton, Fla., United States of America; and U.S. Pat. Nos. 4,235,871; 4,551,482; 6,197,333; and 6,132,766, each of which is incorporated herein by reference in its entirety. Temperature-sensitive liposomes can also be used, for example THERMOSOMES™ as disclosed in U.S. Pat. No. 6,200,598, which is incorporated herein by reference in its entirety. Entrapment of an active agent within liposomes of the presently disclosed subject matter can also be carried out using any conventional method in the art. In preparing liposome compositions, stabilizers such as antioxidants and other additives can be used.

Other lipid carriers can also be used in accordance with the presently disclosed subject matter, such as lipid microparticles, micelles, lipid suspensions, and lipid emulsions. See, e.g., Labat-Moleur et al. (1996) An electron microscopy study into the mechanism of gene transfer with lipopolyamines. Gene Therapy 3:1010-1017; U.S. Pat. Nos. 5,011,634; 6,056,938; 6,217,886; 5,948,767; and 6,210,707, each of which is incorporated herein by reference in its entirety.

In some embodiments, a liposome is a Ceramide Nanoliposome (CNL). CNLs are described in U.S. Pat. No. 8,747,891 and U.S. Patent Application Publication No. 2019/0031756, each of which is incorporated herein in its entirety. In some embodiments, the CNL encompasses the one or more short chain ceramides (including but not limited to C6 ceramides) and one or more chemotherapeutically active agents.

Delivery time frames can be provided according to a desired treatment approach. By way of example and not limitation, the first delivery vehicle can deliver substantially all of the provided active agent within 24 hours after administration wherein the second delivery vehicle can deliver a certain much smaller amount within the first 24 hours, first 3 days, first week, and substantially all within the first 2, 3, 4, 5, 6, or 7 weeks, as desired. Thus, the duration of the delivery can be altered with the chemistry of the delivery vehicle.

The delivery vehicles can comprise nano-, submicron-, and/or micron-sized particles. In some embodiments, the delivery vehicles are about 50 nm to about 1 μm in their largest dimensions. Thus, in some embodiments the delivery vehicle can comprise a nanoparticle, a microparticle, or any combination thereof. As used herein, the terms “nano”, “nanoscopic”, “nanometer-sized”, “nanostructured”, “nanoscale”, and grammatical derivatives thereof are used synonymously and interchangeably and mean nanoparticles and nanoparticle composites less than or equal to about 1,000 nanometers (nm) in diameter. Similarly, the terms “micro”, “microscopic”, “micrometer-sized”, “microstructured”, “microscale”, and grammatical derivatives thereof are used synonymously and interchangeably and mean microparticles and microparticle composites that are larger than 1,000 nanometers (nm) but less than about 5, 10, 25, 50, 100, 250, 500, or 1000 micrometers in diameter.

The term “delivery vehicle” as used herein thus denotes a carrier structure which is biocompatible with and sufficiently resistant to chemical and/or physical destruction by the environment of use such that a sufficient amount of the delivery vehicles remain substantially intact after deployment at a site of interest. If the active agent is to enter a cell, tissue, or organ in a form whereby it is adsorbed to the delivery vehicle, the delivery vehicle must also remain sufficiently intact to enter the cell, tissue, or organ. Biodegradation of the delivery vehicle is permissible upon deployment at a site of interest.

As used herein, the term “biodegradable” means any structure, including but not limited to a nanoparticle, which decomposes or otherwise disintegrates after prolonged exposure to physiological conditions. To be biodegradable, the structure should be substantially disintegrated within a few weeks after introduction into the body.

Biodegradable biocompatible polymers can be used in drug delivery systems (Soppimath et al., (2001) Biodegradable polymeric nanoparticles as drug delivery devices. J Controlled Release 70:1-20; Song et al. (1997) Formulation and characterization of biodegradable nanoparticles for intravascular local drug delivery. J Controlled Release 43:197-212; U.S. Patent Application Publication Nos. 2011/0104069, 2013/0330279, 2018/0078657, 2019/0091280, and 2020/0038452, and U.S. Pat. Nos. 7,332,586; 7,901,711; 8,137,697; 8,449,915; and 8,663,599, each of which is incorporated herein by reference in its entirety). The biodegradability and biocompatibility of poly(lactic acid) (PLA), poly(lactide-co-glycolide) (PLGA), and polyanhydrides (PAH) have been demonstrated. Some of the advantages of these materials include administration in high concentrations of the drug locally with low systemic levels, which reduces systemic complications and allergic reactions (Calhoun et al. (1997) Treatment of osteomyelitis with a biodegradable antibiotic implant. Clin Orthopaed Related Res 341:206-214). Additionally, no follow-up surgical removal is required once the drug supply is depleted (Mandal et al. (2002) Poly(D,L-lactide-co-glycolide) encapsulated poly(vinyl alcohol) hydrogel as a drug delivery system. Pharmaceut Res 19:1713-1719). Biodegradation occurs by simple hydrolysis of the ester backbone in aqueous environments such as body fluids. The degradation products are then metabolized to carbon dioxide and water (de Faria et al. (2005) Preparation and Characterization of Poly(D,L-Lactide) (PLA) and Poly(D,L-Lactide)-Poly(Ethylene Glycol) (PLA-PEG) Nanocapsules Containing Antitumoral Agent Methotrexate. Macromol Symp 229:228-233). Several techniques have been developed to prepare nanoparticles loaded with a broad variety of drugs using PLGA and to some extent with PAH (Lamprecht et al. (1999) Biodegradable monodispersed nanoparticles prepared by pressure homogenization-emulsification. Intl J Pharmaceut 184:97-105; Astete et al. (2006) Synthesis and characterization of PLGA nanoparticles. J Biomater Sci Polymer Edn 17:247-289; Hans et al. (2002) Synthesis and characterization of mPEG-PLA prodrug micelles. Solid State Mater Sci 6:319-327., 2002; Kumar et al. (2004) Preparation and characterization of cationic PLGA nanospheres as DNA carriers. Biomaterials 25:1771-1777; Laurencin et al. (2001) Poly(lactide-co-glycolide)/hydroxyapatite delivery of BMP-2-producing cells: a regional gene therapy approach to bone regeneration. Biomaterials 22:1271-1277; Gonsalves et al. (1998) Synthesis and surface characterization of functionalized polylactide copolymer microparticles. Biomaterials 19:1501-1505; et al. (2001) Preparation of PLGA nanoparticles containing estrogen by emulsification—diffusion method. Colloids Surf A 182:123-130).

In some embodiments, the composition can comprise a pharmaceutically acceptable carrier, diluent, or excipient. As used herein, the term “pharmaceutically acceptable” and grammatical variations thereof, as it refers to compositions, carriers, diluents and reagents, means that the materials are capable of administration to or upon a vertebrate subject without the production of undesirable physiological effects such as nausea, dizziness, gastric upset, fever and the like. In some embodiments, the “pharmaceutically acceptable” refers to pharmaceutically acceptable for use in human beings.

Compositions in accordance with the presently disclosed subject matter generally comprise an amount of the desired delivery vehicle (which can be determined on a case-by-case basis), admixed with an acceptable pharmaceutical diluent or excipient, such as a sterile aqueous solution, to give an appropriate final desired concentration in accordance with the dosage information set forth herein, and/or as would be apparent to one of ordinary skill in the art upon a review of the instant disclosure, with respect to the antibiotic. Such formulations will typically include buffers such as phosphate buffered saline (PBS), or additional additives such as pharmaceutical excipients, stabilizing agents such as BSA or HSA, or salts such as sodium chloride. Such components can be chosen with the preparation of composition for local, and particularly topical, administration in mind.

### III.B. Formulations

The compositions of the presently disclosed subject matter can be administered in any formulation or route that would be expected to deliver the compositions to the subjects and/or target sites present therein.

The compositions of the presently disclosed subject matter comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject. For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, cytokines and other immunomodulating compounds.

### III. C. Routes of Administration

By way of example and not limitation, suitable methods for administering a composition in accordance with the methods of the presently disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, inhalation, intratracheal installation, surgical implantation, transdermal delivery, local injection, intranasal delivery, and hyper-velocity injection/bombardment. Where applicable, continuous infusion can enhance drug accumulation at a target site (see e.g., U.S. Pat. No. 6,180,082, which is incorporated herein by reference in its entirety). In some embodiments, a composition comprising a nanoparticle and/or an exosome is administered orally.

Thus, exemplary routes of administration include parenteral, enteral, intravenous, intraarterial, intracardiac, intrapericardial, intraosseal, intracutaneous, subcutaneous, intradermal, subdermal, transdermal, intrathecal, intramuscular, intraperitoneal, intrasternal, parenchymatous, oral, sublingual, buccal, inhalational, and intranasal. The selection of a particular route of administration can be made based at least in part on the nature of the formulation and the ultimate target site where the compositions of the presently disclosed subject matter are desired to act. In some embodiments, the method of administration encompasses features for regionalized delivery or accumulation of the compositions at the site in need of treatment. In some embodiments, the compositions are delivered directly into the site to be treated.

### III.D. Dose

An effective dose of a composition of the presently disclosed subject matter is administered to a subject in need thereof. An “effective amount” or a “therapeutic amount” is an amount of a composition sufficient to produce a measurable response. Exemplary responses include biologically or clinically relevant responses in subjects such as but not limited to an improvement in a symptom. Actual dosage levels of the compositions of the presently disclosed subject matter can be varied so as to administer an amount of the composition that is effective to achieve the desired response for a particular subject. The selected dosage level will depend upon the activity of the composition, the route of administration, combination with other drugs or treatments, the severity of the disease, disorder, and/or condition being treated, and the condition and prior medical history of the subject being treated. However, it is within the skill of the art to start doses of the compositions of the presently disclosed subject matter at levels lower than required to achieve the desired therapeutic effect and to gradually increase the dosage until the desired effect is achieved. The potency of a composition can vary, and therefore an “effective amount” can vary. However, using the methods described herein, one skilled in the art can readily assess the potency and efficacy of a composition of the presently disclosed subject matter and adjust the regimen accordingly.

As such, after review of the instant disclosure, one of ordinary skill in the art can tailor the dosages to an individual subject, taking into account the particular formulation, method of administration to be used with the composition, and particular disease, disorder, and/or condition treated or biologically relevant outcome desired. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art.

### III.E. Compositions for Uses in the Methods of the Presently Disclosed Subject Matter

Also provided are compositions for use in the methods of the presently disclosed subject matter. As such, in some embodiments the presently disclosed subject matter also provides compositions for use in a method for treating a disease, disorder, and/or condition associated with undesirable cellular proliferation, for use in a method for increasing total ceramide levels in a cell, for use in increasing a ratio of a long chain ceramide to a very long chain ceramide in a cell, for use in enhancing apoptosis of a cell in which apoptosis is desirable, for use in prognosing a subject with a disease, disorder, and/or condition associated with undesirable cellular proliferation with respect to a treatment and for use in increasing sensitivity of a drug-resistant tumor and/or cancer cell to a chemotherapeutic. In some embodiments, the disease, disorder, and/or condition associated with undesirable cellular proliferation is a tumor and/or a cancer, and thus in some embodiments the compositions are for use in treating a tumor and/or a cancer, which can be a leukemia, optionally Acute Myeloid Leukemia (AML).

In some embodiments of the compositions for use in the presently disclosed methods, the chemotherapeutic agent is selected from the group consisting of daunorubicin, AraC, venetoclax, ivodesinib, enasidenib, midostaurin, gilteritinib, decitabine, azacitadine, a histone deacetylase (HDAC) inhibitor, an epigenetic regulator, a histone demethylase inhibitor, or any combination or subcombination thereof.

In some embodiments of the compositions for use in the presently disclosed methods, the composition comprises venetoclax in combination with one or more of daunorubicin, AraC, ivodesinib, enasidenib, midostaurin, gilteritinib, decitabine, azacitadine, a histone deacetylase (HDAC) inhibitor, an epigenetic regulator, and a histone demethylase inhibitor.

In some embodiments of the compositions for use in the presently disclosed methods, the composition comprises, consists essentially of, or consists of venetoclax and AraC, venetoclax and decitabine, or venatoclax and azacitadine.

In some embodiments of the compositions for use in the presently disclosed methods, the composition comprises a ceramide nanoliposome (CNL) that is associated with decitibine, azacitadine, AraC, venetoclax, or any combination or subcombination thereof.

In some embodiments of the compositions for use in the presently disclosed methods, the composition comprises AraC, venetoclax, and one or more short chain ceramides.

In some embodiments of the compositions for use in the presently disclosed methods, the long chain ceramide is a C16 and/or a C18 ceramide.

In some embodiments of the compositions for use in the presently disclosed methods, the very long chain ceramide is a C24 ceramide, optionally a C24:1 ceramide.

In some embodiments of the compositions for use in the presently disclosed methods, contacting a cell with the a composition of the presently disclosed subject matter increases a C16 and/or C18 ceramide to C24 ceramide ratio in the cell. In some embodiments, contacting a cell with a composition of the presently disclosed subject matter increases a C16 ceramide to C24 ceramide ratio, a C18 ceramide to C24 ceramide ratio, or both in the cell. In some embodiments, contacting a cell with a composition of the presently disclosed subject matter decreases a C16 ceramide to C24 ceramide ratio, a C18 ceramide to C24 ceramide ratio, or both in the cell. In some embodiments, the C24 ceramide is a C24:1 ceramide.

### EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Introduction to the Examples

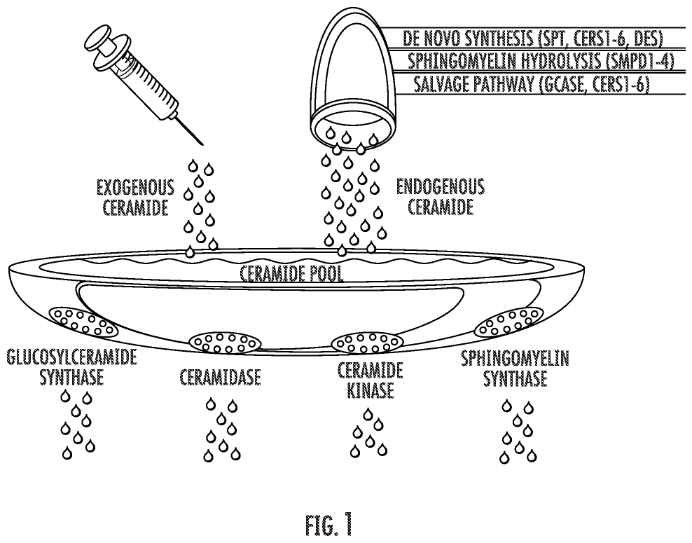

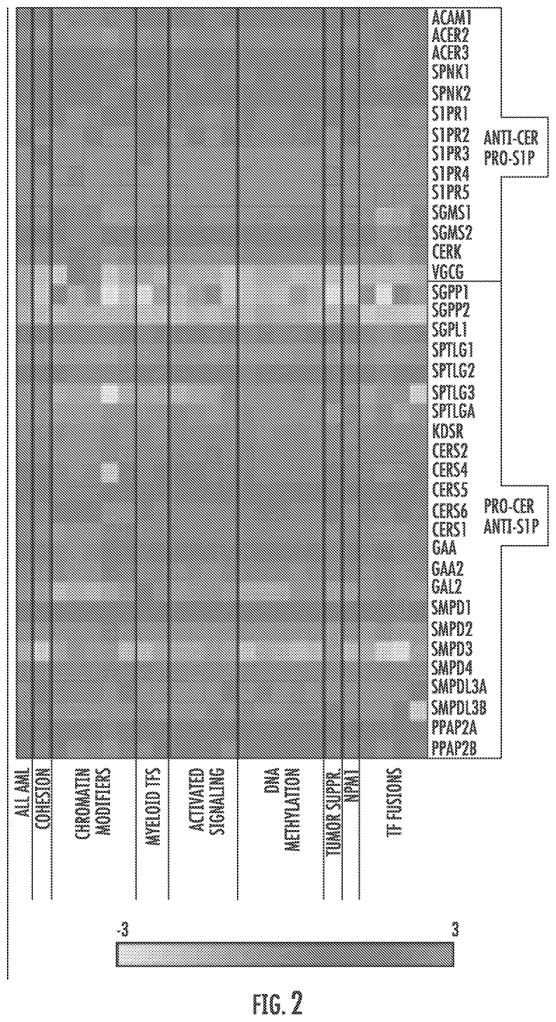

Sphingolipid Metabolism and AML. Sphingolipids are essential for formation of cellular membranes and maintaining structural integrity of the cell. However, sphingolipid metabolism also produces bioactive signaling molecules that regulate critical processes like cell survival and differentiation. Ceramide, sphingosine, and sphingosine 1-phosphate (S1P) represent the bioactive core of the complex sphingolipid synthetic and inter-conversion pathways (see FIG. 1). The fatty acid component of ceramide is cleaved by a ceramidase to produce sphingosine, which is subsequently phosphorylated by sphingosine kinase to produce S1P. Dysregulation and increased flux through this pathway plays a key role in regulating cell survival and response to therapy in multiple cancers. Ceramide accumulation induces apoptosis and other cell death mechanisms, while formation of S1P promotes cell survival through various mechanisms, including receptor-dependent and -independent signaling pathways. This balance is tightly regulated by the enzymes involved in the formation and breakdown of ceramide. When disrupted, cells normally destined for death can expand and lead to disease. In fact, the presently disclosed analyses of TCGA gene expression data revealed widespread alterations in AML of the genes that regulate ceramide metabolism to establish a pro-S1P/anti ceramide state (FIG. 2). As disclosed herein, it is possible to exploit this imbalance as a unique and promising opportunity to target essential biochemical dependencies that represent novel therapeutic approaches.

Recent studies by others have explored multiple therapeutic approaches to manipulate S1P and ceramide levels in AML. For example, treatment of AML cells with the sphingosine analog FTY720 rapidly induced ceramide-mediated apoptosis. Sphingosine kinase 1 is overexpressed in AML, and its inhibition led to reduced levels of the Mcl-1 survival protein and caspase-dependent cell death. Aberrant signaling induced by the FLT3-ITD mutation repressed the production of pro-death ceramide, and the ceramide analog LCL-461 promoted death of AML cells. These studies provide a proof of principle that targeting widespread sphingolipid vulnerabilities in AML creates therapeutic opportunities. However, the use of these ceramide metabolites have never been used as predictive and therapeutic biomarkers for cancers.

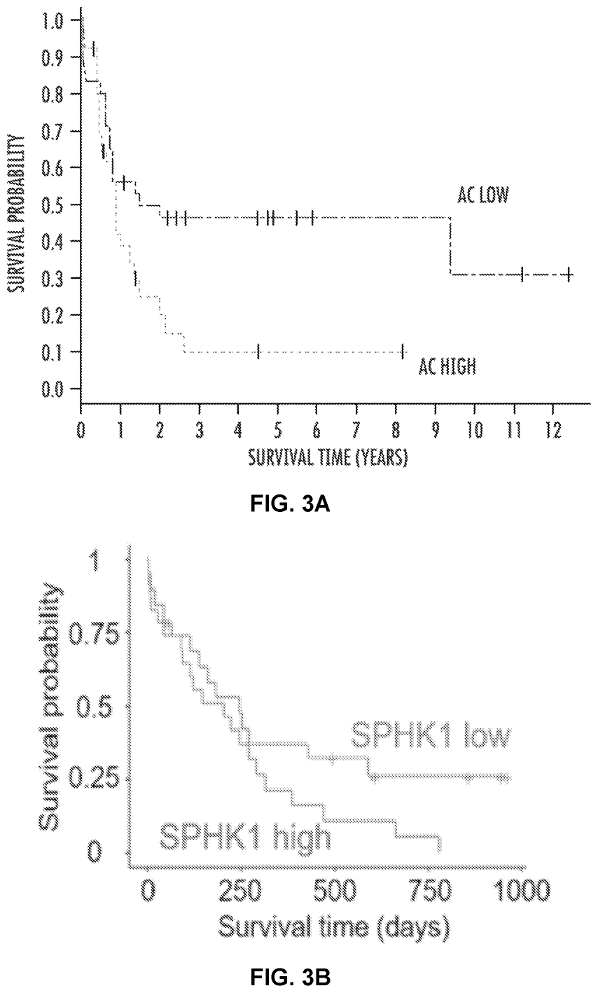

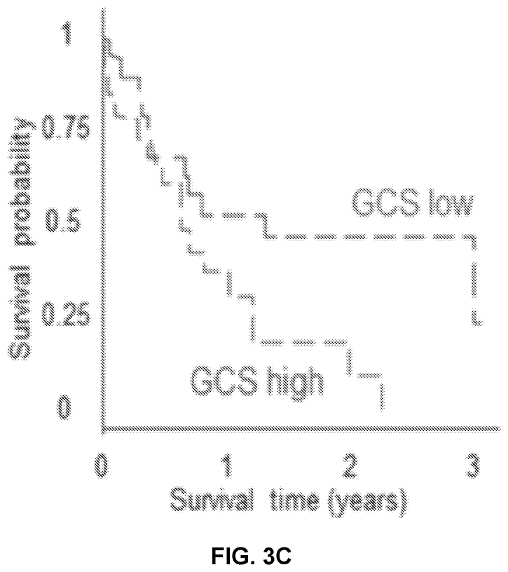

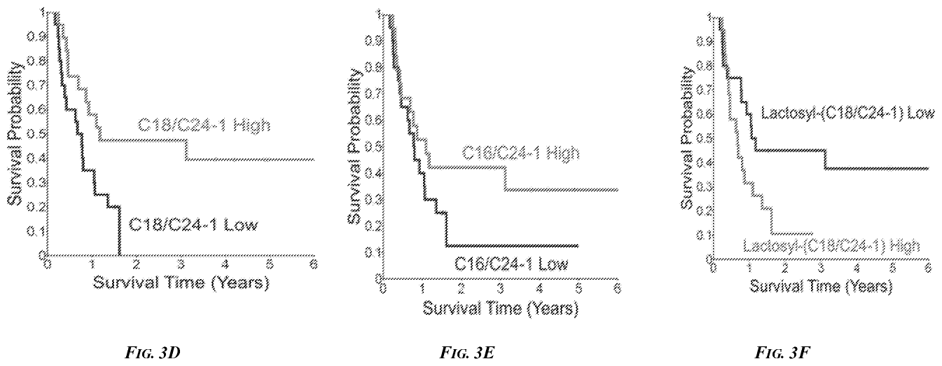

Ceramide ratios as predictive and therapeutic biomarkers for leukemias. It has previously been shown that sphingolipid metabolism is dysregulated in AML and represents a promising target for therapy. That increased expression and/or activity of sphingolipid metabolizing enzymes that metabolize ceramide into less apoptotic lipids (acid ceramidase, sphingosine kinase 1, glucosylceramide synthase) correlate with overall survival in AML has also been demonstrated (FIGS. 3A-3C). Not appreciated previously, however, is the surprising discovery that individual ceramide lipid levels do not correlate with patient outcomes, but that specific lipid ratios do. Specifically, as disclosed herein, whether specific sphingolipid metabolite ratios correlate with survival was investigated by analyzing 95 AML patients prior to induction therapy. Interestingly, individual ceramide species did not correlate with survival, but the ratio of C16 ceramide (FIG. 3D) and C18 ceramide (FIG. 3E) to C24:1 ceramide correlated with progression-free survival (PFS). Also of interest, a low C18/C24:1 lactosylceramide ratio correlated with improved survival (FIG. 3F). Also disclosed herein is the observation that elevated levels of sphingomyelin and glucosylceramides correlate with reduced complete remission. This could reflect increased metabolism of more pro-apoptotic ceramides to less apoptotic metabolites. While a C16/C24 ceramide ratio is used as a biomarker for cardiovascular diseases, this is the first use in cancer. Taken together, the present disclosure represents the first putative lipid ratio that can serve as a predictive or diagnostic biomarker for AML. The instant disclosure further represents the first indication that C16 C18/C24 C24:1 ceramide ratios and not individual lipid levels correlate with overall survival in any cancer.

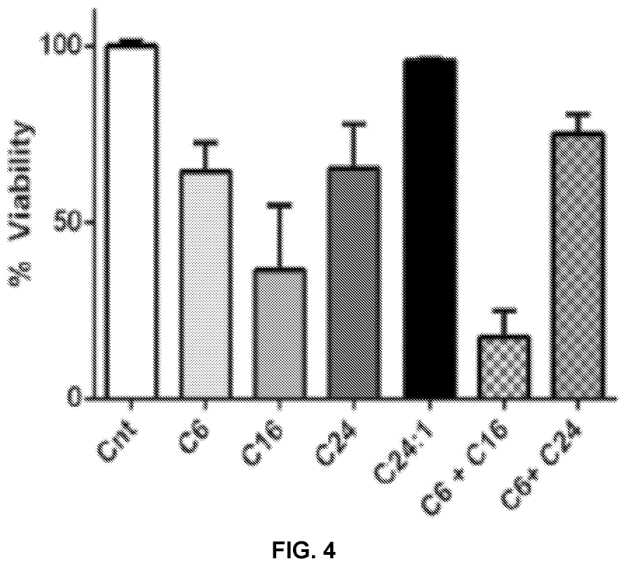

To understand the mechanism by which the ratios of ceramide species can correlate with overall AML survival, the viability of MOLM-14 AML cells in culture was examined by MTS assay. As disclosed herein, increased C16 and C18 ceramides facilitated cell death signaling more so than very long chain ceramides in AML. Specifically, disclosed herein is the discovery that C16 but not C24 ceramide species potentiated CNL-induced cell death in MOLM-14 cells (see FIG. 4).

If these unique ceramide ratios correlated with the response of leukemias to therapies was also investigated. Many conventional chemotherapeutics induce cell death through ceramide generation. Increasing evidence points to the generation of ceramides with C16 and C18 fatty acids, through the specificity of individual ceramide synthases (CerS), may be more apoptotic than C20-C24 ceramides. For example, CerS6 (C16 ceramide generating) has been shown to mediate cell death. Also, Flt3ITD suppresses CerS1 (C18 ceramide generating) and the reactivation of this pathway through Flt3 inhibition leads to cell death.

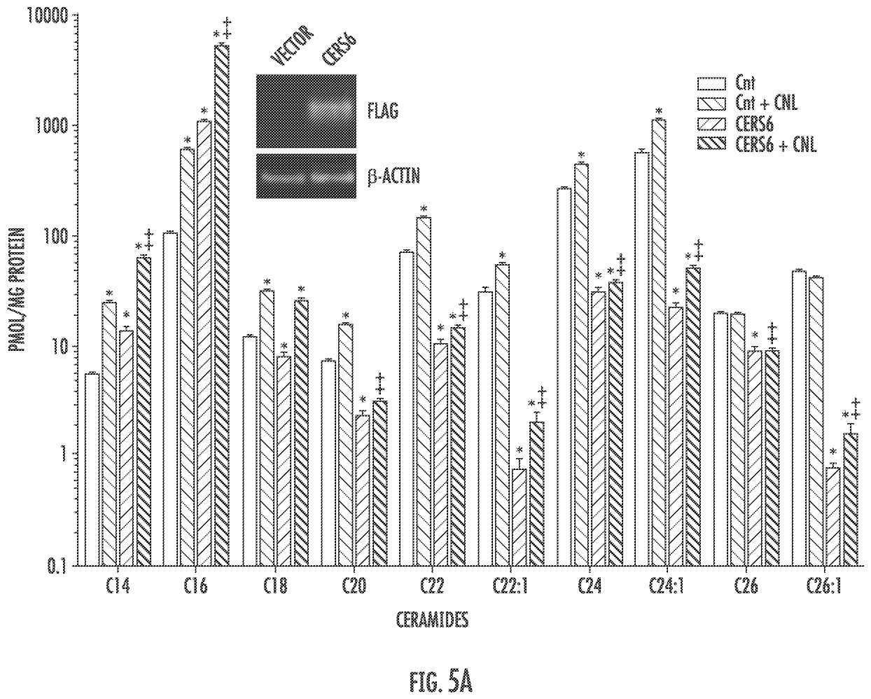

However, ceramide ratios have never been appreciated as potential therapeutic biomarkers. Therefore, the effect of conventional AML chemotherapy (AraC, venetoclax) in the presence or absence of exogenous short chain ceramides (delivered in a nontoxic nanoliposome vehicle, the ceramide nanoliposome, CNL; see e.g., U.S. Pat. No. 8,747,891 or U.S. Patent Application Publication No. 2019/0031756, each of which is incorporated herein in its entirety) on endogenous ceramide levels was tested (see FIG. 5A). All three drugs significantly elevated ceramide levels as single agents and in combination. In the presence of all three drugs, total ceramide levels were dramatically increased. Interestingly, and quite unexpectantly, CNL amplified the ratio of pro-apoptotic C16 and C18 ceramide over less apoptotic ceramides (C16/C24:1 ratio shown in FIG. 5B) in the presence of conventional chemotherapy for AML.

CerS6 overexpressing MOLM-14 cells were generated and analyzed. These cells showed significantly elevated C16 ceramide with reductions in CerS2-generated (C20-26) ceramides (FIG. 5A). The cell death response was next assessed in response to AraC, venetoclax, and/or CNL (FIG. 5B). It was determined that cell death induced by venetoclax and AraC was significantly elevated in the presence of CNL in CerS6 overexpressing cells, implicating that increasing the C16 ceramide to C24:1 ceramide ratio improved efficacy.

Taken together, these data suggested that AraC and venetoclax increases C6 ceramide reacylation to C16 and C18 ceramides, at the expense of C24 ceramides in a CerS- dependent mechanism to improve therapeutic efficacy. These data further validated the findings that the ratio of C16 ceramide (FIG. 3D) or C18 ceramide (FIG. 3E) to C24:1 ceramide in primary AML samples correlated with progression-free survival (PFS). Overall, all data provided validates ceramide ratios as predictive and therapeutic biomarkers of leukemias.

### Materials and Methods for the Examples

Reagents: Ceramide and all other lipids were purchased from Avanti Polar Lipids (Alabaster, Ala., United States of America. Antibodies specific for Mcl-1 (Catalog No. 94296), Bcl-2 (Catalog No. 2872), survivin (Catalog No. 2808), pChk1 (Catalog No. 12302), pChk2 (Catalog No. 2197), p-ERK1/2 (Catalog No. 14095), and XIAP1 (Catalog No. 14334) were purchased from Cell Signaling Technology, Inc. (Danvers, Mass., United States of America). β-actin antibodies were purchased from Sigma-Aldrich (St. Louis, Mo., United States of America; Catalog No. A-5441). DAPI were purchased from Sigma-Aldrich. Venetoclax, Ara-C, and DMSO were purchased from Fisher Scientific (Waltham, Mass., United States of America).

Cell culture: Cells were cultured in suspension in the following media (Gibco™ brand, available from Thermo Fisher Scientific, Waltham, Mass., United States of America). OCI-AML-2: MEM-alpha 20% 10% FBS, OCI-AML-3: MEM-alpha 20% FBS, KG-1: IMDM 20% FBS. MOLM-14: RPMI-1640 with 10% FBS.

MTS Cell Viability Assay: Cytotoxicity of nanoliposomal ceramide in AML cells was measured by plating 15,000 cells/well into 96-well plates followed by growth for 24 hours in a humidified 37° C. cell culture incubator in the presence of ghost liposomes, CNL or other drugs for 24 hours in 10% serum medium. After 24 h, MTS/PMS mixture was added and adsorption was read at 560 nM using Bio-Tek plate reader.

Western blotting: Whole cell lysates were prepared by dissolving cells in FOCUS™ Extraction Buffer III (G-Biosciences, St. Louis, Mo., United States of America; Catalog No. 786-222) containing protease inhibitors (Thermo Fisher Scientific Catalog No. A32955) for 10-15 minutes on ice with occasional shaking. After centrifugation for 15 minutes at 13-14000×g at 10° C. supernatants were mixed with one-third of a volume of NUPAGE™ LDS 4× Sample Buffer (Thermo Fisher Scientific) containing 5% 2-mercaptoethanol. Protein samples were prepared by heating at 37° C. for 10 min after the addition of denaturing sample buffer. Proteins were separated using SDS-PAGE on a 4-12% gel (Thermo Fisher Scientific) and transferred to Immobilon membrane (Bio-Rad). After 15 minute blocking in 1% Casein Blocker in TBS (Thermo Fisher Scientific, Catalog No. 37532), membranes were incubated with the primary antibody overnight at 4° C., washed, incubated with the horseradish peroxidase-conjugated secondary antibody or IR800 infrared dye conjugated secondary antibody (LI-COR Biosciences, Lincoln, Nebr., United States of America), and then washed again. Primary and secondary antibodies were to diluted in 0.05% TBS/Tween-20 containing 0.05% Casein Blocker. Protein bands were visualized using a commercially available chemiluminescence kit (Thermo Fisher Scientific) or by infrared laser scanner using ChemiXX6 G:box imager (Syngene, Frederick, Md., United States of America).

Flow Cytometry: Cells were plated at 24-well tray at 660,000 per well in 2 milliliters of media and CNL or other drugs were added. Twenty-four hours later, 100-microliters cell aliquot was transferred to 96-well tray and mixed with 50 microliters of growth media containing 9 uM DAPI. After 10 minutes incubation at room temperature, cells were analyzed with Attune flow cytometer (Thermo Fisher Scientific). The percentage of cells positive for DAPI staining was calculated using FlowJo software (FlowJo LLC, Ashland, Oreg., United States of America).

Lipidomics: Lipids were extracted from cell lysates and analyzed on an Acquity I-Class/Xevo TQ-S Micro IVD System (Waters Corporation, Milford, Mass., United States of America) as described previously (Pearson et al. (2020) Ceramide Analogue SACLAC Modulates Sphingolipid Levels and MCL-1 Splicing to Induce Apoptosis in Acute Myeloid Leukemia. Mol Cancer Res 18(3):352-363). Mass spectrometry peaks were compared to internal standards, and all data are represented as picomoles of lipid per milligram of protein.

### Example 1

### CNL Augments the Efficacy of Venetoclax and Ara-C in Cultured Cell and Patient Cell Models

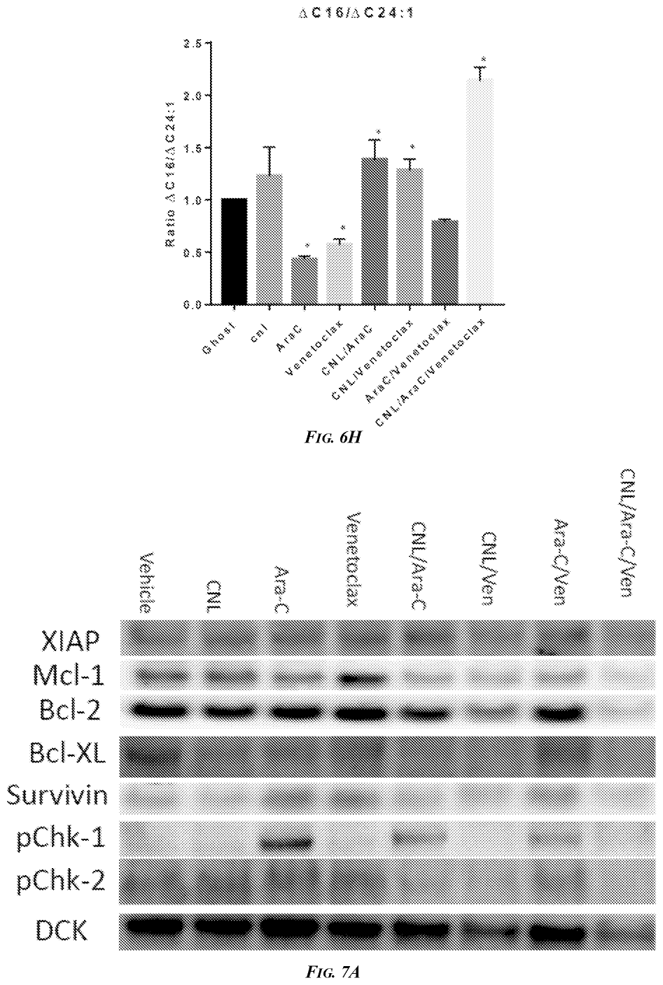

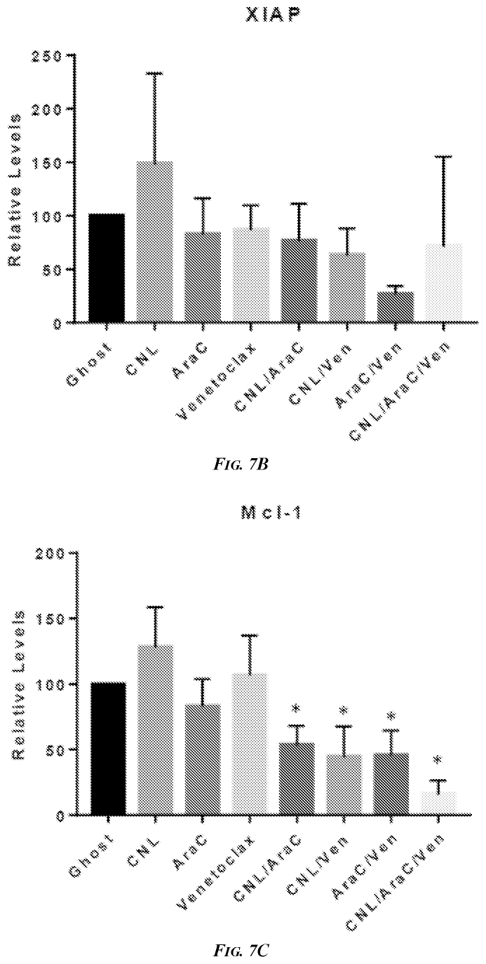

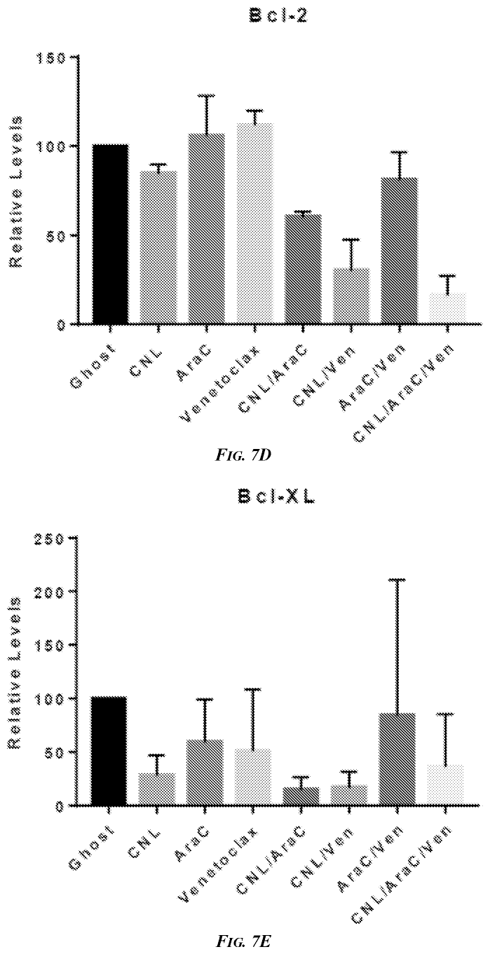

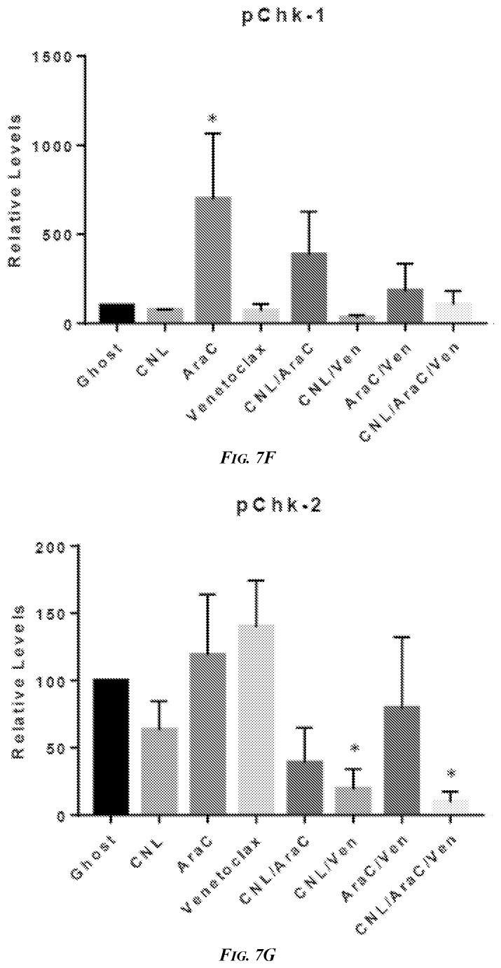

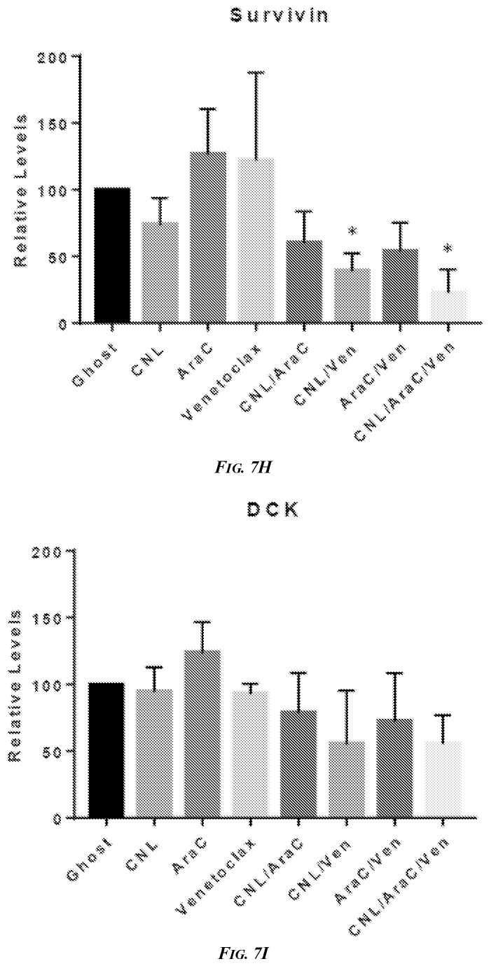

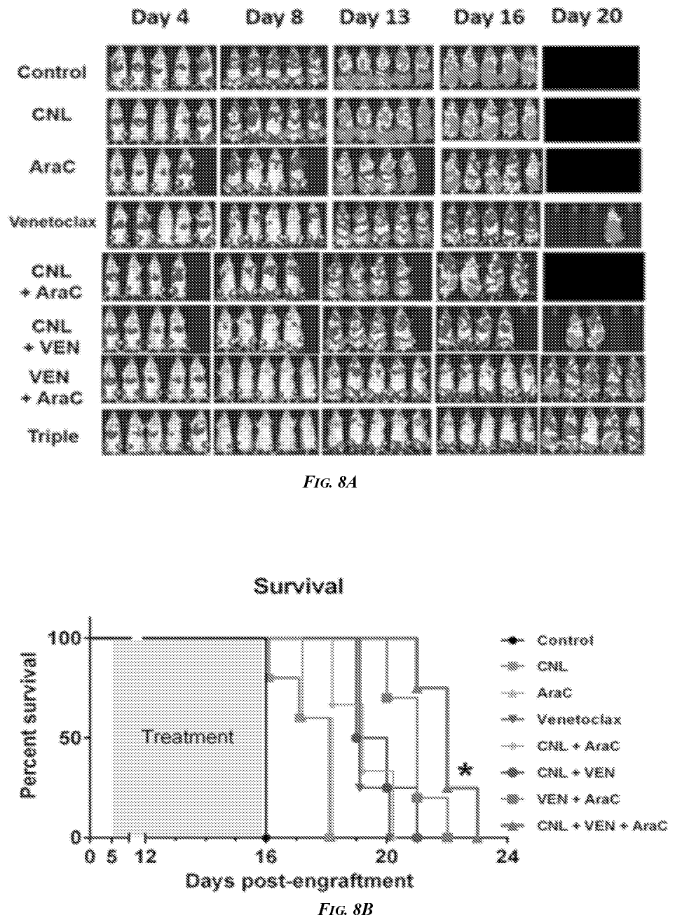

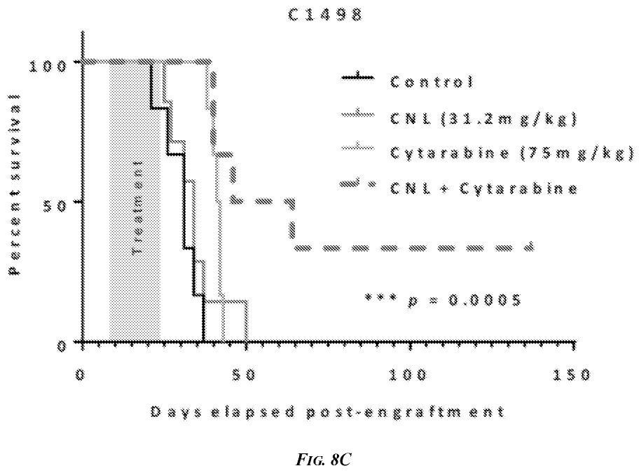

Cell viability MTS assays were performed in three AML cells lines: OCI-AML2, KG-1, AND MOLM-14 to determine their sensitivities to co-treatment with CNL, Ara-C, and Venetoclax. Table 1 summarized these cell lines, their sources, and the known driver mutations that are present in the cell lines. The cells lines were treated with concentrations that had been determined to be less than the EC₅₀ so any synergistic or additive effects could be observed. While CNL was moderately effective in all three cell lines, the triple combination treatment was characterized by a synergistic decrease in cell viability in KG-1 and MOLM-14 (14 and 22% respectively; FIGS. 7B and 7C).

TABLE 1
Descriptions of Cell Lines Employed

| Cell line | Source | Known Driver Mutations |
|---|---|---|
| OCI-AML-2 | peripheral blood of a 65-male with | DNMT3A R635W |
| | acute myeloid leukemia | (MLL-MLLT4; MLL-AF6) |
| | | gene fusion |
| KG-1 | bone marrow of a 59-male | FGFR1OP2-FGFR |
| | erythroleukemia that developed into | |
| | acute myeloid leukemia | |
| MOLM-14 | peripheral blood of a 20-male with | FLT3-ITD |
| | acute myeloid leukemia AML | (MLL-MLLT3; MLL-AF9) |
| | | fusion |

To confirm these cell culture experiments, primary AML patient samples were employed in a colony formation assay to measure sensitivity to the drug combinations. In the two patient samples assayed, the CNL/Ara-C and CNL/Venetoclax treatments were able to decrease the number of colonies formed (FIG. 7D). Of note, the combination of CNL/AraC/Venetoclax (CAV) performed significantly better than any of the dual combinations (p≤0.04 Mann-Whitney test).

As shown in Table 1, Despite differential driver mutations for each of the AML cell lines, the CAV treatment regimine was equally efficatious in all cell lines.

### ExampleE 2

### Venetoclax and AraC Metabolize CNL in Pro-Apoptotic Ceramide Species

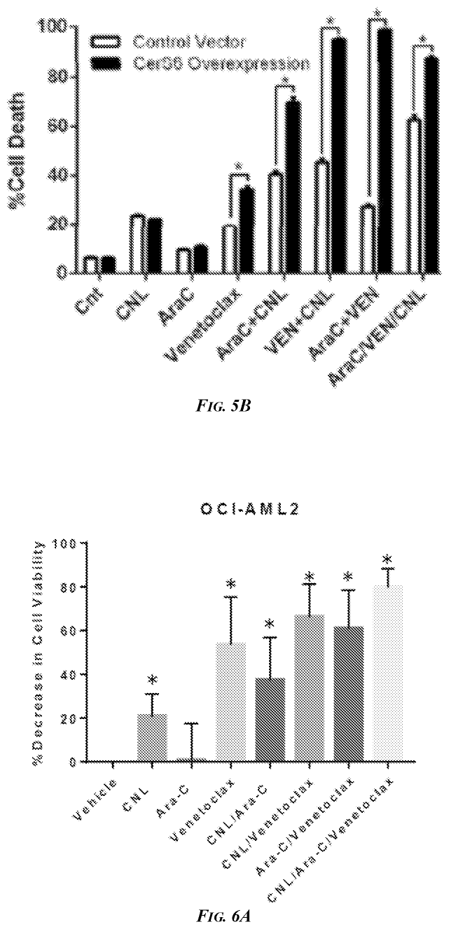

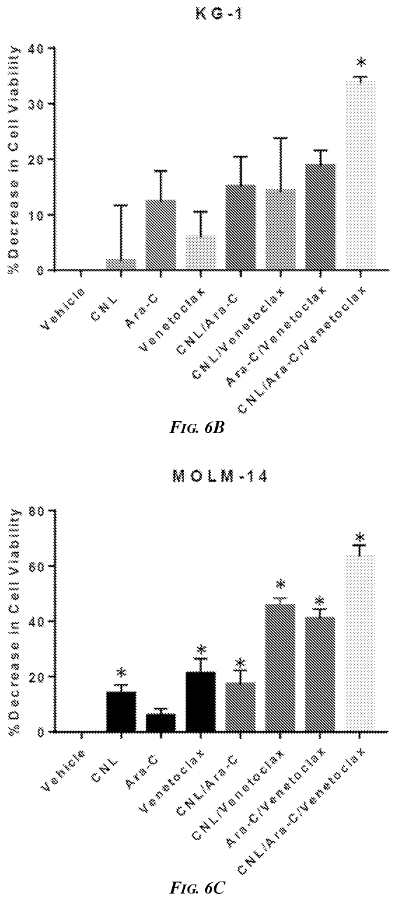

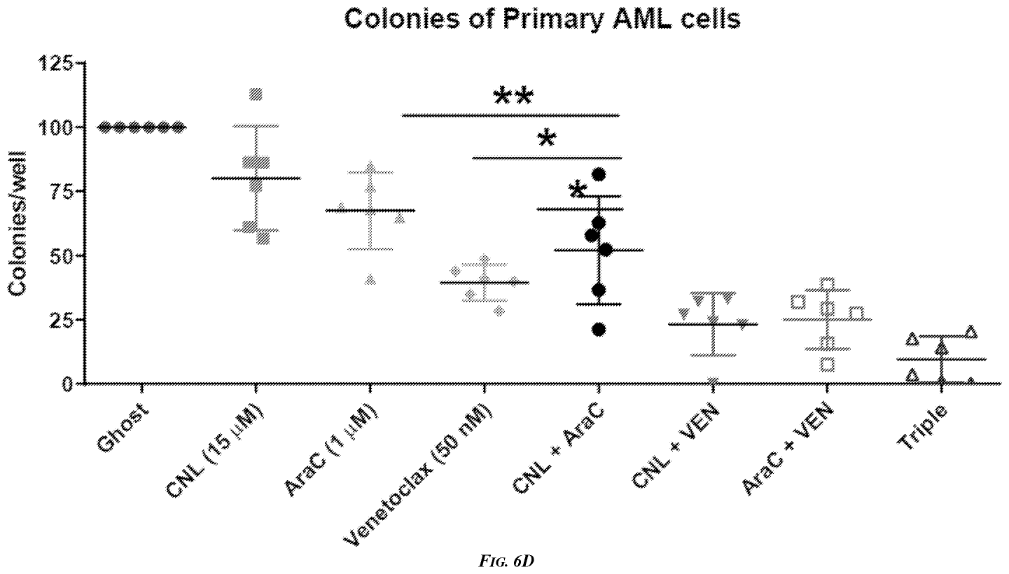

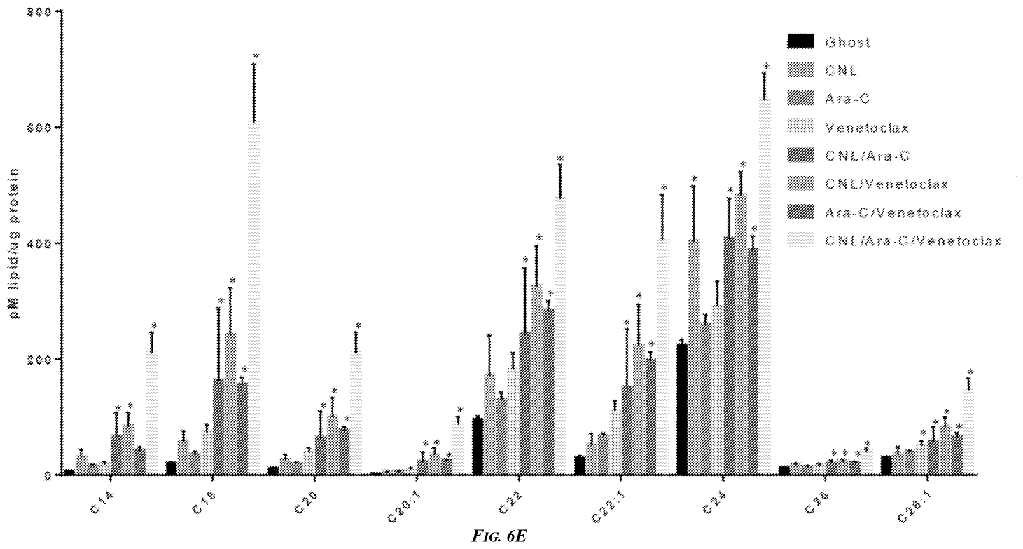

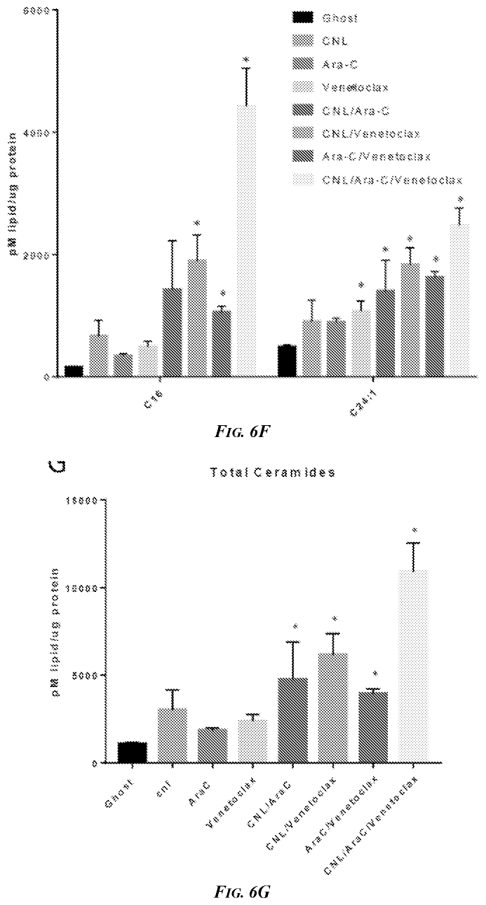

To determine potential metabolic mechanism underlying the efficacy of combinatorial CNL/AraC/Venetoclax (CAV) treatment, lipids were harvested to measure the effect of Venetoclax and AraC upon CNL metabolism by LC/MS. Cells were treated individually or in combination with CNL, Ara-C, and/or Venetoclax for 24 hrs. CNL alone was able to increase levels of all the ceramide species (FIGS. 6E and 6F). Both Venetoclax and Ara-C were able to raise ceramide levels with co-treatment of CNL (FIGS. 6E and 6F). Interestingly, treatment with all 3 led to a marked increase in individual ceramide species (FIGS. 6E and 6F) as well as total ceramide levels (FIG. 6G). Of note, the increase was highly significant for the highly apoptotic C16 ceramide species (FIG. 6F). Elevations in less toxic species, including C22-C24, where less pronounced. This lead to an increase in the C16/C24 ratio, which might support a pro-mitogenic response (FIG. 6H).

### Example 3

### CNL Diminishes Anti-Apoptotic Peptides and Pro-Mitogenic Kinases

Whether CNL could diminish drug-resistant survival mechanism induced by either Venetoclax or Ara-C was also investigated. Venetoclax resistant cells have been shown to upregulate the anti-apoptotic proteins myeloid leukemia cell differentiation protein (Mcl-1; Phillips et al. (2015) Loss in MCL-1 function sensitizes non-Hodgkin's lymphoma cell lines to the BCL-2-selective inhibitor venetoclax (ABT-199). Blood Cancer J 5(11):e368.). Western blots of MOLM-14 cells treated with the drug combinations showed a small but not statistically significant increase in Mcl-1 and XIAP with Venetoclax, but not Ara-C (FIGS. 7A-7C). This increase was decreased to baseline upon co-treatment with CNL (FIGS. 7A-7C). Interestingly, Ara-C with Venetoclax treatment reduced Mcl-1 expression below baseline (FIGS. 7A and 7C). In addition, Venetoclax as well as Ara-C independently increased expression levels of the apoptosis inhibitor, Survivin. (FIGS. 7A and 7H). In both cases, treatment with CNL was able to decrease Survivin levels below baseline (FIGS. 7A and 7H). In a similar vein, Ara-C has been shown to increase levels of phosphorylation of the checkpoint inhibitors Chk1 and Chk2, allowing continuation of the cell cycle (Dai & Grant (2010) New insights into checkpoint kinase 1 in the DNA damage response signaling network. Clin Cancer Res 16(2):376-383; Takagaki et al. (2005) Role of Chk1 and Chk2 in Ara-C-induced differentiation of human leukemia K562 cells. Genes Cells 10(2):97-106). CNL, as well as Venetoclax treatment reduced Ara-C induced protein levels of pChk1 and pChk2 (FIGS. 7A, 7F, and 7G). Taken together, CNL reduced drug-resistant, pro-mitogenic signaling induced by Venetolax and Ara-C.

### Example 4

### CNL/Ara-C/Venetoclax Co-Treatment is Highly Effective in RR Animal Models

Given these positive in vitro studies, we sought to determine if the addition of CNL would increase the efficacy of Ara-C/Venetoclax treatment using AML animal models. We initially utilized the immune-compromised NRG mice engrafted with MOLM-13 cells expressing luciferase. Mice were engrafted, treated with the various drug combinations, and disease burden measured by live animal luminescence and Kaplan-Meier curve. The combination of CAV treatment was the most efficacious compared to all other groups, including the standard of care, Ara-C+Venetoclax group, at reducing tumor burden (FIG. 9A) and increasing survival (FIG. 9B). We next confirmed these data utilizing the syngeneic C1498-luc mouse AML model. C1498 cells are a de novo AML cell line from a C57-blkJ mouse. One important constraint of utilizing this line is that it is insensitive to Venetoclax treatment. Given this, no Venetoclax groups were included. C57-blkJ mice were injected with C1498 AML cells transduced to express luciferase. After engraftment was confirmed, mice were treated with the different drug combinations for 7 days. Both Ara-C or CNL alone had a moderate increase in survival. The combination therapy, however, had a median survival of 55 days with a large percentage surviving over 130 days when the mice were sacrificed.

### Discussion of the Examples

Given the current low rates of durable response and high toxicity of current AML therapies, new drug candidates are needed. We have shown previously that CNL is efficacious in AML with MDS related changes (Barth et al. (2019) Sphingolipid metabolism determines the therapeutic efficacy of nanoliposomal ceramide in acute myeloid leukemia. Blood Adv 3(17):2598-2603). We now sought to see if CNL would be potent when added to the current mainline therapy of Venetoclax and low dose Ara-C (LDAC; Lachowiez et al. (2020) Venetoclax in acute myeloid leukemia—current and future directions. Leuk Lymphoma 61(6):1313-1322). We showed a synergistic effect in two common AML cell lines when CAV was given together further strengthening this hypothesis. It appears this is due to the combinatorial therapy increasing total ceramide levels (FIG. 7H). In the two most common ceramides, C16 and C24:1, we see C16 ceramide increases to a much larger extent than C24:1 (FIG. 7G). The increase observed in the C16/C24 ceramide ratio in the CAV treatment group could be of potentially great interest. It has been reported that the C16/C24:1 ceramide ratio in plasma has been shown to an independent marker of cardiovascular mortality (Peterson et al. (2018) Ceramide Remodeling and Risk of Cardiovascular Events and Mortality. J Am Heart Assoc 7(10):e007931). This could potentially indicate a new biomarker for AML. Previously, studies have shown that ceramide can alter the expression and/or post translational modifications of multiple proteins involved in cell growth, apoptosis, and other forms of cell death. We have shown that CNL therapy can decrease clinically relevant resistant mechanism to Venetoclax and Ara-C (FIGS. 7A-7I) This increase in sensitivity is further born out in patient samples and in vivo AML, models. Taken together, we provide compelling in vitro and in vivo data demonstrating that CNL augments the effectiveness of the combination of Ara-C and Venetoclax, thus providing the rationale to pursue clinical trials.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.

It will be understood that various details of the presently disclosed subject matter can be changed without departing from the scope of the presently disclosed subject matter. Furthermore, the foregoing description is for the purpose of illustration only, and not for the purpose of limitation.

1. A method for treating a disease, disorder, and/or condition associated with undesirable cellular proliferation, the method comprising administering to a subject in need thereof a therapeutically effective amount of a composition comprising, consisting essentially of, or consisting of a chemotherapeutic agent and a short chain ceramide.

2. The method of claim 1, wherein the disease, disorder, and/or condition associated with undesirable cellular proliferation is a tumor and/or a cancer.

3. The method of claim 2, wherein the tumor and/or the cancer is a leukemia, optionally Acute Myeloid Leukemia (AML).

4. The method of claim 1, wherein the chemotherapeutic agent is selected from the group consisting of daunorubicin, AraC, venetoclax, ivodesinib, enasidenib, midostaurin, gilteritinib, decitabine, azacitadine, a histone deacetylase (HDAC) inhibitor, an epigenetic regulator, a histone demethylase inhibitor, or any combination or subcombination thereof.

5. The method of claim 4, wherein the composition comprises venetoclax in combination with one or more of daunorubicin, AraC, ivodesinib, enasidenib, midostaurin, gilteritinib, decitabine, azacitadine, a histone deacetylase (HDAC) inhibitor, an epigenetic regulator, and a histone demethylase inhibitor.

6. The method of claim 1, wherein the composition comprises, consists essentially of, or consists of venetoclax and AraC, venetoclax and decitabine, or venatoclax and azacitadine.

7. The method of claim 6, wherein the composition comprises a ceramide nanoliposome (CNL) that is associated with decitibine, azacitadine, AraC, venetoclax, or any combination or subcombination thereof.

8. The method of claim 1, wherein the composition comprises AraC, venetoclax, and one or more short chain ceram ides.

9. A method for increasing total ceramide levels in a cell, the method comprising contacting the cell with an effective amount of a composition comprising, consisting essentially of, or consisting of one or more short chain ceramides and one or more chemotherpaeutic agents, optionally wherein the one or more chemotherspeutic agents are selected from the group consisting of daunorubicin, AraC, venetoclax, ivodesinib, enasidenib, midostaurin, gilteritinib, decitabine, azacitadine, a histone deacetylase (HDAC) inhibitor, an epigenetic regulator, a histone demethylase inhibitor, and combinations thereof.

10. A method for increasing a ratio of a long chain ceramide to a very long chain ceramide in a cell, the method comprising contacting the cell with an effective amount of a composition comprising, consisting essentially of, or consisting of one or more short chain ceramides and one or more chemotherapeutic agent.

11. The method of claim 10, wherein the long chain ceramide is a C16 and/or a C18 ceramide.

12. The method of claim 10, wherein the very long chain ceramide is a C24 ceramide.

13. The method of claim 10, wherein the one or more chemotherapeutic agents are selected from the group consisting of daunorubicin, AraC, venetoclax, ivodesinib, enasidenib, midostaurin, gilteritinib, decitabine, azacitadine, a histone deacetylase (HDAC) inhibitor, an epigenetic regulator, a histone demethylase inhibitor, or any combination or subcombination thereof.

14. The method of claim 13, wherein the composition comprises venetoclax in combination with one or more of daunorubicin, AraC, ivodesinib, enasidenib, midostaurin, gilteritinib, decitabine, azacitadine, a histone deacetylase (HDAC) inhibitor, an epigenetic regulator, and a histone demethylase inhibitor.

15. The method of claim 1, wherein the composition comprises, consists essentially of, or consists of venetoclax and AraC, venetoclax and decitabine, or venatoclax and azacitadine.

16. The method of claim 10, wherein the composition comprises a ceramide nanoliposome (CNL) that is associated with decitibine, azacitadine, AraC, venetoclax, or any combination or subcombination thereof.

17. The method of claim 10, wherein the composition comprises one or more short chain ceramides and AraC or venetoclax or both AraC and venetoclax.

18. The method of claim 10, wherein the cell is a tumor and/or a cancer cell, optionally a leukemia cell, further optionally an AML cell.

19. The method of claim 18, wherein the method further comprises contacting the cell with a further anti-leukemia therapeutic agent.

20. A method for enhancing apoptosis of a cell in which apoptosis is desirable, the method comprising contacting the cell with a composition comprising, consisting essentially of, or consisting of a chemotherapeutic agent in combination with one or more small chain ceramides.

21. The method of claim 20, wherein the chemotherapeutic agent is selected from the group consisting of daunorubicin, AraC, venetoclax, ivodesinib, enasidenib, midostaurin, gilteritinib, decitabine, azacitadine, a histone deacetylase (HDAC) inhibitor, an epigenetic regulator, a histone demethylase inhibitor, or any combination or subcombination thereof.

22. The method of claim 21, wherein the composition comprises venetoclax in combination with one or more of daunorubicin, AraC, ivodesinib, enasidenib, midostaurin, gilteritinib, decitabine, azacitadine, a histone deacetylase (HDAC) inhibitor, an epigenetic regulator, and a histone demethylase inhibitor.

23. The method of claim 20, wherein the composition comprises, consists essentially of, or consists of venetoclax and AraC, venetoclax and decitabine, or venatoclax and azacitadine.

24. The method of claim 20, wherein the composition comprises a ceramide nanoliposome (CNL) that is associated with decitibine, azacitadine, AraC, venetoclax, or any combination or subcombination thereof.

25. The method of claim 20, wherein the composition comprises one or more short chain ceramides and AraC or venetoclax or both AraC and venetoclax.

26. The method of claim 20, wherein the cell is a tumor and/or a cancer cell, optionally a leukemia cell, further optionally an AML cell.

27. The method of claim 20, wherein the contacting increases a C16 and/or C18 ceramide to C24 ceramide ratio in the cell.

28. The method of claim 27, wherein the contacting increases a C16 ceramide to C24 ceramide ratio, a C18 ceramide to C24 ceramide ratio, or both in the cell.

29. The method of claim 27, wherein the contacting decreases a C16 ceramide to C24 ceramide ratio, a C18 ceramide to C24 ceramide ratio, or both in the cell.

30. The method of claim 289, wherein the C24 ceramide is a C24:1 ceramide.

31. A method for prognosing a subject with a disease, disorder, and/or condition associated with undesirable cellular proliferation with respect to a treatment, the method comprising determining a ratio of long chain ceramide to very long chain ceramide in the subject, wherein the ratio is indicative of progression-fee survival (PFS), improved overall survival, complete remission, or any combination thereof in the subject.

32. The method of claim 31, wherein the disease, disorder, and/or condition associated with undesirable cellular proliferation is a tumor and/or a cancer.

33. The method of claim 32, wherein the tumor and/or the cancer is a leukemia, optionally Acute Myeloid Leukemia (AML).

34. The method of claim 31, wherein the long chain ceramide is selected from the group consisting of a C16 ceramide, a C18 ceramide, or any combination thereof.

35. The method of claim 31, wherein the very long chain ceramide is a C24 ceramide, optionally a C24:1 ceramide.

36. The method of claim 31, wherein a high C16 and/or C18 to C24 ceramide ratio in the subject is indicative of improved survival.

37. The method of claim 31, wherein a low C16 and/or C18 to C24 lactosylceramide ratio in the subject is indicative of improved survival.

38. The method of claim 1, wherein the composition comprises a ceramide nanoliposome (CNL).

39. A method for increasing sensitivity of a drug-resistant tumor and/or cancer cell to a chemotherapeutic, the method comprising contacting the drug-resistant tumor and/or cancer cell with a therapeutically effective amount of a composition comprising, consisting essentially of, or consisting of one or more small chain ceramides in combination with the chemotherapeutic, wherein the sensitivity of the drug-resistant tumor and/or cancer cell to the chemotherapeutic is increased relative to the sensitivity of the drug-resistant tumor and/or cancer cell prior to the contacting.

40. The method of claim 39, wherein the chemotherapeutic is venetoclax, AraC, decitabine, azacitadine, an HDAC inhibitor, an epigenetic regulator, a histone demethylase inhibitor, or any combination of subcombination thereof.

41. The method of claim 39, wherein the contacting increases a ratio of C16 and/or C18 ceramide to C24 ceramide in the drug-resistant tumor and/or cancer cell.

42. The method of claim 39, wherein the tumor and/or cancer cell is present in a subject and the composition is administered to the subject via a route and in an amount sufficient to increase the sensitivity of the drug-resistant tumor and/or cancer cell to the chemotherapeutic.

43. The method of claim 1, wherein the subject is a mammal, optionally a human.

44. A composition, optionally a pharmaceutical composition, comprising, consisting essentially of, or consisting of one or more short chain ceramides and one or more chemotherapeutically active agents.

45. The composition of claim 44, wherein the composition further comprises one or more pharmaceutically acceptable carriers, diluents, and/or excipients, optionally wherein the composition is pharmaceutically acceptable for use in a human.

46. The composition of claim 44, wherein the one or more short chain ceramides are saturated C6 ceramides, monosaturated C6 ceram ides, or any combination thereof.

47. The composition of claim 44, wherein the one or more chemotherapeutically active agents are selected from the group consisting of venetoclax, AraC, decitabine, azacitadine, an HDAC inhibitor, an epigenetic regulator, a histone demethylase inhibitor, and combinations thereof.

48. A composition for use in a method for treating a disease, disorder, and/or condition associated with undesirable cellular proliferation, for use in increasing total ceramide levels in a cell, for use in increasing a ratio of a long chain ceramide to a very long chain ceramide in a cell, for use in enhancing apoptosis of a cell in which apoptosis is desirable, for prognosing a subject with a disease, disorder, and/or condition associated with undesirable cellular proliferation with respect to a treatment, and/or for use in increasing sensitivity of a drug-resistant tumor and/or cancer cell to a chemotherapeutic, the composition comprising, consisting essentially of, or consisting of a chemotherapeutic agent in combination with one or more small chain ceramides.

49. The composition for use of claim 48, wherein the disease, disorder, and/or condition associated with undesirable cellular proliferation is a tumor and/or a cancer, optionally a leukemia, further optionally Acute Myeloid Leukemia (AML).

50. The composition for use of claim 48, wherein the chemotherapeutic agent is selected from the group consisting of daunorubicin, AraC, venetoclax, ivodesinib, enasidenib, midostaurin, gilteritinib, decitabine, azacitadine, a histone deacetylase (HDAC) inhibitor, an epigenetic regulator, a histone demethylase inhibitor, or any combination or subcombination thereof.

51. The composition for use of claim 48, wherein the composition comprises venetoclax in combination with one or more of daunorubicin, AraC, ivodesinib, enasidenib, midostaurin, gilteritinib, decitabine, azacitadine, a histone deacetylase (HDAC) inhibitor, an epigenetic regulator, and a histone demethylase inhibitor.

52. The composition for use of claim 48, wherein the composition comprises, consists essentially of, or consists of venetoclax and AraC, venetoclax and decitabine, or venatoclax and azacitadine.

53. The composition for use of claim 48, wherein the composition comprises a ceramide nanoliposome (CNL) that is associated with decitibine, azacitadine, AraC, venetoclax, or any combination or subcombination thereof.

54. The composition for use of claim 48, wherein the composition comprises AraC, venetoclax, and one or more short chain ceramides.

55. The composition for use of claim 48, wherein the long chain ceramide is a C16 and/or a C18 ceramide.

56. The composition for use of claim 48, wherein the very long chain ceramide is a C24 ceramide, optionally a C24:1 ceram ide.

57. The composition for use of claim 48, wherein the composition increases a C16 and/or C18 ceramide to C24 ceramide ratio in the cell.

58. The composition for use of claim 48, wherein the composition increases a C16 ceramide to C24 ceramide ratio, a C18 ceramide to C24 ceramide ratio, or both in the cell.

59. The composition for use of claim 48, wherein the composition decreases a C16 ceramide to C24 ceramide ratio, a C18 ceramide to C24 ceramide ratio, or both in the cell.

60. The composition for use of claim 48, wherein the C24 ceramide is a C24:1 ceramide.
